(19) United States
(12) Patent Application Publication
(10) Pub. No.: US 2024/0112407 A1
(43) Pub. Date: Apr. 4, 2024

(54) SYSTEM, METHODS, AND STORAGE MEDIUMS FOR RELIABLE URETEROSCOPES AND/OR FOR IMAGING

(71) Applicants: Canon U.S.A., Inc., Melville, NY (US); The Brigham and Women's Hospital Inc., Boston, MA (US)

(72) Inventors: Fumitaro Masaki, Brookline, MA (US); Takahisa Kato, Brookline, MA (US); Nobuhiko Hata, Waban, MA (US); Satoshi Kobayashi, Boston, MA (US); Franklin King, Boston, MA (US)

(21) Appl. No.: 18/477,081

(22) Filed: Sep. 28, 2023

Related U.S. Application Data
(60) Provisional application No. 63/377,983, filed on Sep. 30, 2022.
(60) Provisional application No. 63/378,017, filed on Sep. 30, 2022.
(60) Provisional application No. 63/383,210, filed on Nov. 10, 2022.

(51) Int. Cl. G06T 19/00; A61B 1/00; A61B 1/307; A61B 18/26; G06T 7/00; G06T 7/50

(52) U.S. Cl. CPC G06T 19/00; A61B 1/000094; A61B 1/307; A61B 18/26; G06T 7/0012; G06T 7/50; G06T 2207/10068; G06T 2207/10081; G06T 2207/30084; G06T 2210/41

(57) ABSTRACT

One or more devices, systems, methods, and storage mediums for performing imaging and/or visualization and/or for performing lithotripsy are provided herein. Examples of applications include imaging, evaluating, and diagnosing biological objects, such as, but not limited to, for ureteral, Gastro-intestinal, cardio, bronchial, and/or ophthalmic applications, and being obtained via one or more optical instruments, such as, but not limited to, optical probes, catheters, ureteroscopes, endoscopes, and bronchoscopes. Techniques provided herein also improve image processing and efficiency and provide reliable imaging techniques that may be used for one or more applications, including, but not limited to, ureteroscopy and lithotripsy, while reducing mental and physical burden and improving ease of use.

### CROSS-REFERENCE TO RELATED APPLICATION(S)

This application relates, and claims priority, to U.S. Patent Application Ser. No. 63/378,017, filed Sep. 30, 2022, the disclosure of which is incorporated by reference herein in its entirety, and this application relates, and claims priority, to U.S. Patent Application Ser. No. 63/377,983, filed Sep. 30, 2022, the disclosure of which is incorporated by reference herein in its entirety, and to U.S. Patent Application Ser. No. 63/383,210, filed Nov. 10, 2022, the disclosure of which is incorporated by reference herein in its entirety.

### FIELD OF THE DISCLOSURE

The present disclosure generally relates to imaging and, more particularly, to a continuum robot apparatus, method, and storage medium that operate to image a target, object, or specimen (such as, but not limited to, a calyx, a kidney, ureters, tissue, etc.). One or more ureteroscopic, endoscopic, medical, camera, catheter, or imaging devices, systems, and methods and/or storage mediums for use with same, are discussed herein. One or more devices, methods, or storage mediums may be used for medical applications and, more particularly, to steerable, flexible medical devices that may be used for or with guide tools and devices in medical procedures, including, but not limited to, endoscopes, cameras, catheters, and ureteroscopes.

### BACKGROUND

Ureteroscopy, endoscopy, bronchoscopy, and other medical procedures facilitate the ability to look inside a body. During such a procedure, a flexible medical tool may be inserted into a patient's body, and an instrument may be passed through the tool to examine or treat an area inside the body. A ureteroscope is an instrument to view inside the ureters and kidneys. Catheters and other medical tools may be inserted through a tool channel in the ureteroscope or other imaging device to provide a pathway to a target area in the patient for diagnosis, planning, medical procedure(s), treatment, etc.

Robotic ureteroscopes may be equipped with a tool channel or a camera and biopsy tools, and may insert/retract the camera and biopsy tools to exchange such components. The robotic ureteroscopes may be used in association with a display system and a control system.

An imaging device, such as a camera, may be placed on or in the ureteroscopes to capture images inside the patient, and a display or monitor may be used to view the captured images. The display system may display, on the monitor, an image or images captured by the camera, and the display system may have a display coordinate used for displaying the captured image or images. In addition, the control system may control a moving direction of the tool channel or the camera. For example, the tool channel or the camera may be bent according to a control by the control system. The control system may have an operational controller (such as, but not limited to, a joystick, a gamepad, a controller, an input device, etc.).

Ureteroscopy for transurethral lithotripsy may be performed with a ureteroscope to look for a urinary stone, break apart a urinary stone, and/or remove a urinary stone or urinary stone fragments. In a case where a physician overlooks some of the fragments in the urinary system, the patient may have to retake transurethral lithotripsy procedure again. To avoid the extra procedure, the physician preferably carefully checks all of the locations of the urinary system. However, there is no reliable way to record whether the physician has already checked all of the locations or not. At this point, the physician typically mentally memorizes whether the physician checked all of the locations or not.

Accordingly, it would be desirable to provide one or more imaging devices, systems, methods, and/or storage mediums that address the aforementioned issues while providing a physician, technician, or other practitioner with ways to have a reliable way or ways to know whether a location or locations have been checked for fragments and/or urinary stones already or not.

### SUMMARY

Accordingly, it is a broad object of the present disclosure to provide imaging (e.g., computed tomography (CT), Magnetic Resonance Imaging (MRI), etc.) apparatuses, systems, methods, and storage mediums for providing features that operate to record, display, and/or indicate whether an area or areas have already been inspected or checked (and/or not yet inspected or checked such an area or areas may be unchecked or uninspected) for fragments or urinary stones or not.

One or more embodiments of an information processing apparatus or system may include one or more processors that operate to: obtain a three dimensional image of an object, target, or sample; acquire positional information of an image capturing tool inserted in or into the object, target, or sample; determine, based on the positional information of the image capturing tool and the relative position of the image capturing tool to the object, target, or sample, portions of the object, target, or sample that are a first portion and a second portion, where the first portion corresponds to a portion of the object, target, or sample where the image capturing tool has captured or inspected and the second portion corresponds to a portion of the object, target, or sample where the image capturing tool still is or remains to be captured or to be inspected such that the second portion is uncaptured or uninspected; and display, based on the acquired positional information, the first portion of the three dimensional image of the object, target, or sample with a first expression which is different from a second expression of the second portion of the three dimensional image. In one or more embodiments, the one or more processors further operate to display the second portion and/or the second expression for the second portion along with the first portion and the first expression. In one or more embodiments, the object, target, or sample may be one or more of the following: an anatomy, a kidney, a urinary system, or a portion of a kidney or urinary system. In one or more embodiments, the first portion may correspond to a portion that the image capturing tool has captured or inspected in a Field-of-View (FOV) of the image capturing tool, and the second portion may correspond to a portion that remains to be captured or inspected in the Field-of-View of the image capturing tool. In one or more embodiments, the captured or inspected first portion represents or corresponds to an overlap between an imaging Field-of-View or a view model (e.g., a cone or other geometric shape being used for the model) of the image capturing tool and the surfaces of the object, target, or sample being imaged (e.g., the inner surfaces of a kidney that are located within the FOV or the view model (e.g., the cone or other predetermined or set geometric shape)).

In one or more embodiments, the one or more processors further operate to: receive a predetermined or set acceptable size, or a size within a predetermined or set range, of a missed or uninspected/uncaptured area, the missed or uninspected/uncaptured area being an area or portion that is not captured or inspected, or remains to be captured or inspected, by the image capturing tool; and display a third portion of the three dimensional image of the object, sample, or target with a third expression which is different from both of the expressions of the first portion and the second portion of the three dimensional image, wherein the third portion corresponds to the missed or uninspected/uncaptured area of which size is equal to or less than the predetermined or set acceptable size. In one or more embodiments, the one or more processors may further operate to: receive a predetermined or set acceptable percentage of a completion of a capturing or inspection of the object, target, or sample; and indicate a completion of the capturing or inspection of the object, target, or sample, in a case where the percentage of an area or portion captured or inspected by the image capturing tool is equal to or more than the predetermined or set acceptable percentage.

In one or more embodiments, the one or more processors further operate to: store time information corresponding to a length of time that a particular portion or area is within the Field-of-View of the image capturing tool; and display the three dimensional image of the anatomy with the first expression of the first portion after the image capturing tool has captured or inspected the first portion for a period of time indicated by the stored time information. In one or more embodiments, the stored time information is the accumulated during to have overlap between an imaging Field-of-View of the image capturing tool and the surfaces of the object, target, or sample being imaged (e.g., the inner surfaces of a kidney).

In one or more embodiments, the one or more processors further operate to: acquire the positional information of the image capturing tool based on a shape of the image capturing tool calculated based on a forward kinematics model. In one or more embodiments, the one or more processors further operate to: acquire the positional information of the image capturing tool based on a shape sensor of the image capturing tool. In one or more embodiments, the one or more processors further operate to: acquire the positional information of the image capturing tool based on a positional information detected by an electromagnetic sensor.

In one or more embodiments, the one or more processors further operate to: display, based on a depth map from or based on a captured image captured by the image capturing tool, the three dimensional image of the object, sample, or target. In one or more embodiments, the one or more processors further operate to: display, based on the acquired positional information, the first portion of the three dimensional image of the object, sample, or target with a first color being different from a second color of or used for the second portion of the three dimensional image.

In one or more embodiments, a physician may record and display an area where the physician or other practitioner has already checked to see whether there is a fragment or fragments of a crushed stone or not. By confirming that the physician has completed checking all areas of the kidney, overlooking fragments of the urinary stone may be restrained or avoided. In the end, a possibility of causing complications is reduced, and additional procedures may be avoided. In one or more embodiments, a physician or other practitioner may save time to search for any residuals of one or more fragments of a urinary stone. In one or more embodiments, a physician or other practitioner may check the completeness of a procedure or imaging based on time, or the capturing or inspection may be based on time. In one or more embodiments, the one or more processors may further operate to calculate a viewed area (e.g., the first portion) without using an Electro Magnetic (EM) tracking system, and/or may further operate to calculate a viewed area (e.g., the first portion) using a camera view of the imaging apparatus or system (e.g., a ureteroscope or other imaging apparatus or system) without using an Electro Magnetic (EM) tracking system. In one or more embodiments, the one or more processors may further operate to search for uric acid stones (e.g., at a beginning of a procedure or imaging), and/or the one or more processors may further operate to apply a visualization of a viewed area (e.g., the first portion) to find a uric acid stone (e.g., where a uric acid stone may be invisible by intraoperative fluoroscopy). In one or more embodiments, a physician or other practitioner may confirm the areas that have already been viewed, captured, or inspected during a procedure or imaging.

In one or more embodiments, a method for imaging may include: obtaining a three dimensional image of an object, target, or sample; acquiring positional information of an image capturing tool inserted in or into the object, target, or sample; determining, based on the positional information of the image capturing tool and the relative position of the image capturing tool to the object, target, or sample, portions of the object, target, or sample that are a first portion and a second portion, where the first portion corresponds to a portion of the object, target, or sample where the image capturing tool has captured or inspected and the second portion corresponds to a portion of the object, target, or sample where the image capturing tool still is or remains to be captured or to be inspected such that the second portion is uncaptured or uninspected; and displaying, based on the acquired positional information, the first portion of the three dimensional image of the object, target, or sample with a first expression which is different from a second expression of the second portion of the three dimensional image. In one or more embodiments, the one or more processors further operate to display the second portion and/or the second expression for the second portion along with the first portion and the first expression. In one or more embodiments, the object, target, or sample may be one or more of the following: an anatomy, a kidney, a urinary system, or a portion of a kidney or urinary system. In one or more embodiments, the first portion may correspond to a portion that the image capturing tool has captured or inspected in a Field-of-View of the image capturing tool, and the second portion may correspond to a portion that remains to be captured or inspected in the Field-of-View of the image capturing tool. In one or more embodiments, the one or more processors further operate to: display, based on a depth map from or based on a captured image captured by the image capturing tool, the three dimensional image of the object, sample, or target. In one or more embodiments, the one or more processors further operate to: display, based on the acquired positional information, the first portion of the three dimensional image of the object, sample, or target with a first color being different from a second color of or used for the second portion of the three dimensional image. In one or more embodiments, the first, second, and/or third expressions may be different appearances, patterns, colors, displays of information or data, or other expressions discussed herein.

In one or more embodiments, a non-transitory computer-readable storage medium may store at least one program for causing a computer or processor to execute a method for imaging, where the method may include: obtaining a three dimensional image of an object, target, or sample; acquiring positional information of an image capturing tool inserted in or into the object, target, or sample; determining, based on the positional information of the image capturing tool and the relative position of the image capturing tool to the object, target, or sample, portions of the object, target, or sample that are a first portion and a second portion, where the first portion corresponds to a portion of the object, target, or sample where the image capturing tool has captured or inspected and the second portion corresponds to a portion of the object, target, or sample where the image capturing tool still is or remains to be captured or to be inspected such that the second portion is uncaptured or uninspected; and displaying, based on the acquired positional information, the first portion of the three dimensional image of the object, target, or sample with a first expression which is different from a second expression of the second portion of the three dimensional image. In one or more embodiments, the one or more processors further operate to display the second portion and/or the second expression for the second portion along with the first portion and the first expression. In one or more embodiments, the object, target, or sample may be one or more of the following: an anatomy, a kidney, a urinary system, or a portion of a kidney or urinary system. In one or more embodiments, the first portion may correspond to a portion that the image capturing tool has captured or inspected in a Field-of-View of the image capturing tool, and the second portion may correspond to a portion that remains to be captured or inspected in the Field-of-View of the image capturing tool. In one or more embodiments, the one or more processors further operate to: display, based on a depth map from or based on a captured image captured by the image capturing tool, the three dimensional image of the object, sample, or target. In one or more embodiments, the one or more processors further operate to: display, based on the acquired positional information, the first portion of the three dimensional image of the object, sample, or target with a first color being different from a second color of or used for the second portion of the three dimensional image.

In one or more embodiments, a method for performing lithotripsy may include: obtaining a Computed Tomography (CT) scan; segmenting an object, target, or sample; starting lithotripsy; inserting an Electro Magnetic (EM) sensor into a tool channel; performing registration; starting visualization of a viewing area; moving an imaging apparatus or system or a robotic ureteroscope to search for a urinary stone in an object or specimen; crushing the urinary stone into fragments using a laser inserted into the tool channel; removing the fragments of the urinary stone using a basket catheter; inserting the EM sensor into the tool channel; performing registration; starting visualization of a viewing area; displaying a first portion and a second portion of the viewing area, where the first portion indicates a portion that has been captured or inspected and the second portion indicates a portion that still is or remains to be captured or imaged such that the second portion is uninspected or uncaptured; moving an imaging apparatus or system or a robotic ureteroscope to search for any one or more residual fragments; and, in a case where any residual fragment(s) are found, removing the fragment(s).

In one or more embodiments, a non-transitory computer-readable storage medium may store at least one program for causing a computer or processor to execute a method for performing lithotripsy, where the method may include: obtaining a Computed Tomography (CT) scan; segmenting an object, target, or sample; starting lithotripsy; inserting an Electro Magnetic (EM) sensor into a tool channel; performing registration; starting visualization of a viewing area; moving an imaging apparatus or system or a robotic ureteroscope to search for a urinary stone in an object or specimen; crushing the urinary stone into fragments using a laser inserted into the tool channel; removing the fragments of the urinary stone using a basket catheter; inserting the EM sensor into the tool channel; performing registration; starting visualization of a viewing area; displaying a first portion and a second portion of the viewing area, where the first portion indicates a portion that has been captured or inspected and the second portion indicates a portion that still is or remains to be captured or imaged such that the second portion is uninspected or uncaptured; moving an imaging apparatus or system or a robotic ureteroscope to search for any one or more residual fragments; and, in a case where any residual fragment(s) are found, removing the fragment(s).

In accordance with one or more embodiments of the present disclosure, apparatuses and systems, and methods and storage mediums for performing imaging may operate to characterize biological objects, such as, but not limited to, blood, mucus, tissue, etc.

One or more features and/or embodiments of the present disclosure may be used in clinical application(s), such as, but not limited to, intervascular imaging, intravascular imaging, ureteroscopy, lithotripsy, bronchoscopy, atherosclerotic plaque assessment, cardiac stent evaluation, intracoronary imaging using blood clearing, balloon sinuplasty, sinus stenting, arthroscopy, ophthalmology, ear research, veterinary use and research, etc.

In accordance with at least another aspect of the present disclosure, one or more technique(s) discussed herein may be employed as or along with features to reduce the cost of at least one of manufacture and maintenance of the one or more apparatuses, devices, systems, and storage mediums by reducing or minimizing a number of optical and/or processing components and by virtue of the efficient techniques to cut down cost (e.g., physical labor, mental burden, fiscal cost, time and complexity, reduce or avoid procedure(s), etc.) of use/manufacture of such apparatuses, devices, systems, and storage mediums.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods, and one or more storage mediums using imaging and/or other technique(s) are discussed herein. Further features of the present disclosure will in part be understandable and will in part be apparent from the following description and with reference to the included drawings.

### BRIEF DESCRIPTION OF THE DRAWINGS

Further objectives, features and advantages of the present disclosure will become apparent from the following detailed description when taken in conjunction with the accompanying figures showing illustrative embodiments of the present disclosure.

FIGS. 1 and 2 illustrate at least one example embodiment of an imaging or endoscopic apparatus or system in accordance with one or more aspects of the present disclosure;

FIG. 3 is a schematic diagram showing at least one embodiment console or computer that may be used with one or more correction or adjustment imaging technique(s) in accordance with one or more aspects of the present disclosure;

FIGS. 4A-4C illustrate at least one embodiment example of a continuum robot, imaging apparatus, and/or medical device that may be used with one or more correction or adjustment imaging technique(s) in accordance with one or more aspects of the present disclosure;

FIG. 5 is a schematic diagram showing at least one embodiment an imaging or continuum robot apparatus or system in accordance with one or more aspects of the present disclosure;

FIG. 6 is a flowchart of at least one embodiment of a method for planning an operation of at least one embodiment of a continuum robot apparatus or system in accordance with one or more aspects of the present disclosure;

FIG. 7 is a flowchart of at least one embodiment of a method for painting a 3D model or performing a visualization mode in accordance with one or more aspects of the present disclosure;

FIG. 8 shows a flowchart of at least one embodiment of a method for performing a visualization in accordance with one or more aspects of the present disclosure;

FIG. 9 is a schematic diagram of at least one embodiment of an apparatus or system that may be used for performing a visualization in accordance with one or more aspects of the present disclosure;

FIG. 10 illustrates at least one embodiment of a display or graphical user interface (GUI) views using visualization technique(s) in accordance with one or more aspects of the present disclosure;

FIG. 11 illustrates at least one embodiment of a display or graphical user interface (GUI) views using visualization technique(s) in accordance with one or more aspects of the present disclosure;

FIG. 12 illustrates at least one embodiment of a display or graphical user interface (GUI) views using visualization technique(s) in accordance with one or more aspects of the present disclosure;

FIG. 13 illustrates at least one embodiment of a display or graphical user interface (GUI) views using visualization technique(s) in accordance with one or more aspects of the present disclosure;

FIG. 14 shows a flowchart of at least one embodiment of a method for performing a visualization in accordance with one or more aspects of the present disclosure;

FIG. 15 is a schematic diagram of at least one embodiment of an apparatus or system that may be used for performing a visualization in accordance with one or more aspects of the present disclosure;

FIG. 16 illustrates at least one embodiment of a display or graphical user interface (GUI) views using visualization technique(s) in accordance with one or more aspects of the present disclosure;

FIG. 17 shows a flowchart of at least one embodiment of a method for performing a visualization in accordance with one or more aspects of the present disclosure;

FIG. 18 is a schematic diagram of at least one embodiment of an apparatus or system that may be used for performing a visualization in accordance with one or more aspects of the present disclosure;

FIG. 19 illustrates at least one embodiment of a display or graphical user interface (GUI) views using visualization technique(s) in accordance with one or more aspects of the present disclosure;

FIG. 20 shows a flowchart of at least one embodiment of a method for performing a visualization in accordance with one or more aspects of the present disclosure;

FIG. 21 illustrates a diagram of a continuum robot that may be used with one or more visualization technique(s) or method(s) in accordance with one or more aspects of the present disclosure;

FIG. 22 illustrates a block diagram of at least one embodiment of a continuum robot in accordance with one or more aspects of the present disclosure;

FIG. 23 illustrates a block diagram of at least one embodiment of a controller in accordance with one or more aspects of the present disclosure;

FIG. 24 shows a schematic diagram of an embodiment of a computer that may be used with one or more embodiments of an apparatus or system or one or more methods discussed herein in accordance with one or more aspects of the present disclosure; and

FIG. 25 shows a schematic diagram of another embodiment of a computer that may be used with one or more embodiments of an imaging apparatus or system or methods discussed herein in accordance with one or more aspects of the present disclosure.

### DETAILED DESCRIPTION OF THE PRESENT DISCLOSURE

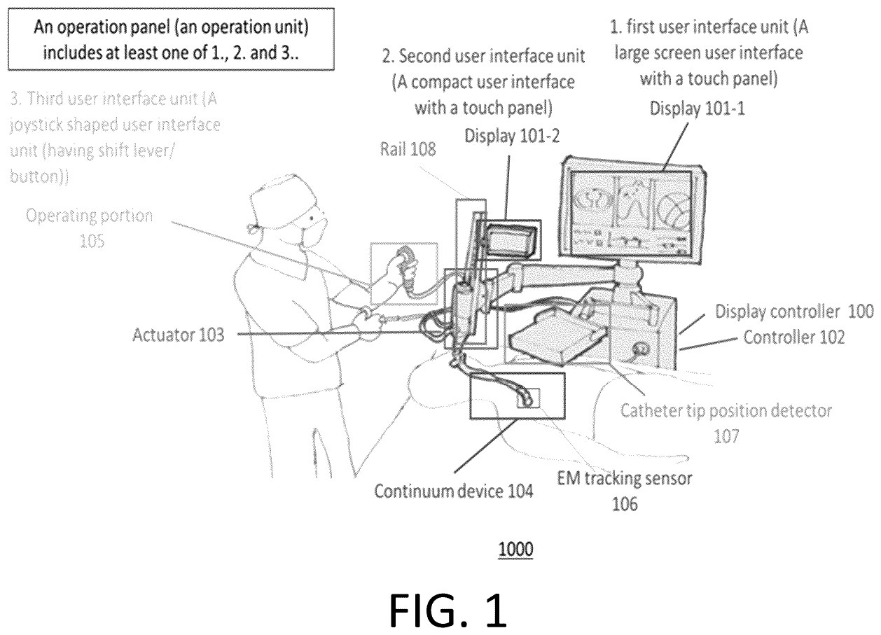

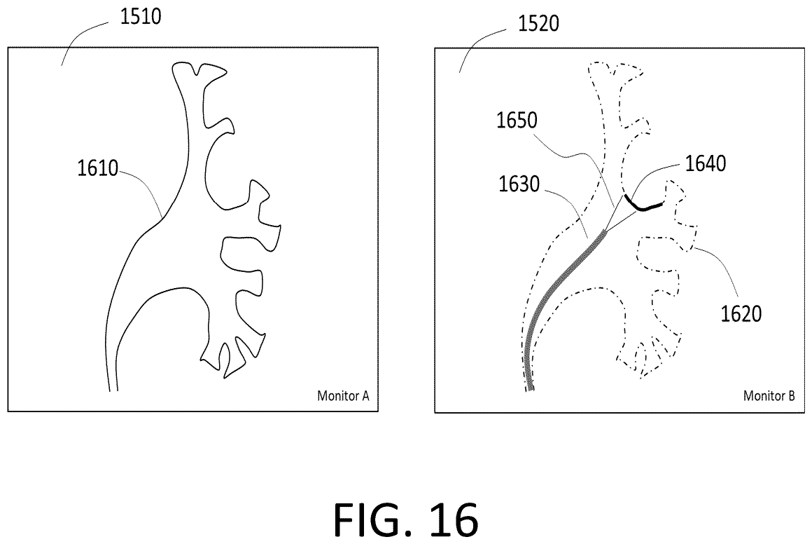

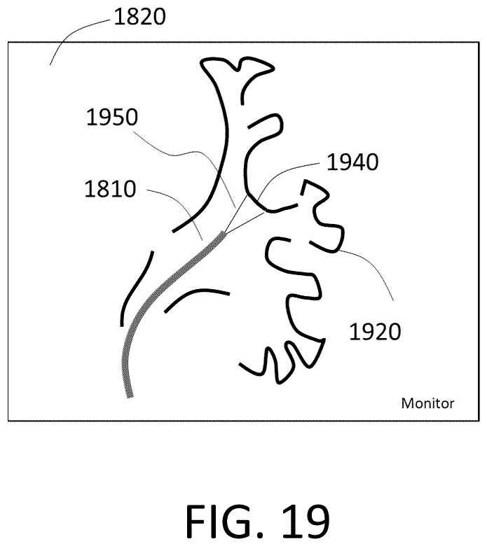

One or more devices, systems, methods, and storage mediums for viewing, imaging, and/or characterizing tissue, or an object or sample, using one or more imaging techniques or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure are described diagrammatically and visually in FIGS. 1 through 25.

In one or more embodiments, a physician may record and display an area where the physician or other practitioner has already checked to see whether there is a fragment or fragments of a crushed stone or not. By confirming that the physician has completed checking all areas of the kidney, overlooking fragments of the urinary stone may be restrained or avoided. In the end, a possibility of causing complications is reduced, and additional procedures may be avoided. In one or more embodiments, a physician or other practitioner may save time to search for any residuals of one or more fragments of a urinary stone. In one or more embodiments, a physician or other practitioner may check the completeness of a procedure or imaging based on time, or the capturing or inspection may be based on time. In one or more embodiments, the one or more processors may further operate to calculate a viewed area (e.g., the first portion) without using an Electro Magnetic (EM) tracking system, and/or may further operate to calculate a viewed area (e.g., the first portion) using a camera view of the imaging apparatus or system (e.g., a ureteroscope or other imaging apparatus or system) without using an Electro Magnetic (EM) tracking system. In one or more embodiments, the one or more processors may further operate to search for uric acid stones (e.g., at a beginning of a procedure or imaging), and/or the one or more processors may further operate to apply a visualization of a viewed area (e.g., the first portion) to find a uric acid stone (e.g., where a uric acid stone may be invisible by intraoperative fluoroscopy). In one or more embodiments, a physician or other practitioner may confirm the areas that have already been viewed, captured, or inspected during a procedure or imaging.

One or more embodiments of an information processing apparatus or system may include one or more processors that operate to: obtain a three dimensional image of an object, target, or sample; acquire positional information of an image capturing tool inserted in or into the object, target, or sample; determine, based on the positional information of the image capturing tool and the relative position of the image capturing tool to the object, target, or sample, portions of the object, target, or sample that are a first portion and a second portion, where the first portion corresponds to a portion of the object, target, or sample where the image capturing tool has captured or inspected and the second portion corresponds to a portion of the object, target, or sample where the image capturing tool still is or remains to be captured or to be inspected such that the second portion is uncaptured or uninspected; and display, based on the acquired positional information, the first portion of the three dimensional image of the object, target, or sample with a first expression which is different from a second expression of the second portion of the three dimensional image. In one or more embodiments, the one or more processors further operate to display the second portion and/or the second expression for the second portion along with the first portion and the first expression. In one or more embodiments, the object, target, or sample may be one or more of the following: an anatomy, a kidney, a urinary system, or a portion of a kidney or urinary system. In one or more embodiments, the first portion may correspond to a portion that the image capturing tool has captured or inspected in a Field-of-View (FOV) of the image capturing tool, and the second portion may correspond to a portion that remains to be captured or inspected in the Field-of-View of the image capturing tool. In one or more embodiments, the captured or inspected first portion represents or corresponds to an overlap between an imaging Field-of-View or a view model (e.g., a cone or other geometric shape being used for the model) of the image capturing tool and the surfaces of the object, target, or sample being imaged (e.g., the inner surfaces of a kidney that are located within the FOV or the view model (e.g., the cone or other predetermined or set geometric shape)).

In one or more embodiments, the one or more processors further operate to: receive a predetermined or set acceptable size, or a size within a predetermined or set range, of a missed or uninspected/uncaptured area, the missed or uninspected/uncaptured area being an area or portion that is not captured or inspected, or remains to be captured or inspected, by the image capturing tool; and display a third portion of the three dimensional image of the object, sample, or target with a third expression which is different from both of the expressions of the first portion and the second portion of the three dimensional image, wherein the third portion corresponds to the missed or uninspected/uncaptured area of which size is equal to or less than the predetermined or set acceptable size. In one or more embodiments, the one or more processors may further operate to: receive a predetermined or set acceptable percentage of a completion of a capturing or inspection of the object, target, or sample; and indicate a completion of the capturing or inspection of the object, target, or sample, in a case where the percentage of an area or portion captured or inspected by the image capturing tool is equal to or more than the predetermined or set acceptable percentage.

In one or more embodiments, the one or more processors further operate to: store time information corresponding to a length of time that a particular portion or area is within the Field-of-View of the image capturing tool; and display the three dimensional image of the anatomy with the first expression of the first portion after the image capturing tool has captured or inspected the first portion for a period of time indicated by the stored time information. In one or more embodiments, the stored time information is the accumulated during to have overlap between an imaging Field-of-View of the image capturing tool and the surfaces of the object, target, or sample being imaged (e.g., the inner surfaces of a kidney).

In one or more embodiments, the one or more processors further operate to: acquire the positional information of the image capturing tool based on a shape of the image capturing tool calculated based on a forward kinematics model. In one or more embodiments, the one or more processors further operate to: acquire the positional information of the image capturing tool based on a shape sensor of the image capturing tool. In one or more embodiments, the one or more processors further operate to: acquire the positional information of the image capturing tool based on a positional information detected by an electromagnetic sensor.

In one or more embodiments, the one or more processors further operate to: display, based on a depth map from or based on a captured image captured by the image capturing tool, the three dimensional image of the object, sample, or target. In one or more embodiments, the one or more processors further operate to: display, based on the acquired positional information, the first portion of the three dimensional image of the object, sample, or target with a first color being different from a second color of or used for the second portion of the three dimensional image.

In one or more embodiments, a physician may record and display an area where the physician or other practitioner has already checked to see whether there is a fragment or fragments of a crushed stone or not. By confirming that the physician has completed checking all areas of the kidney, overlooking fragments of the urinary stone may be restrained or avoided. In the end, a possibility of causing complications is reduced, and additional procedures may be avoided. In one or more embodiments, a physician or other practitioner may save time to search for any residuals of one or more fragments of a urinary stone. In one or more embodiments, a physician or other practitioner may check the completeness of a procedure or imaging based on time, or the capturing or inspection may be based on time. In one or more embodiments, the one or more processors may further operate to calculate a viewed area (e.g., the first portion) without using an Electro Magnetic (EM) tracking system, and/or may further operate to calculate a viewed area (e.g., the first portion) using a camera view of the imaging apparatus or system (e.g., a ureteroscope or other imaging apparatus or system) without using an Electro Magnetic (EM) tracking system. In one or more embodiments, the one or more processors may further operate to search for uric acid stones (e.g., at a beginning of a procedure or imaging), and/or the one or more processors may further operate to apply a visualization of a viewed area (e.g., the first portion) to find a uric acid stone (e.g., where a uric acid stone may be invisible by intraoperative fluoroscopy). In one or more embodiments, a physician or other practitioner may confirm the areas that have already been viewed, captured, or inspected during a procedure or imaging.

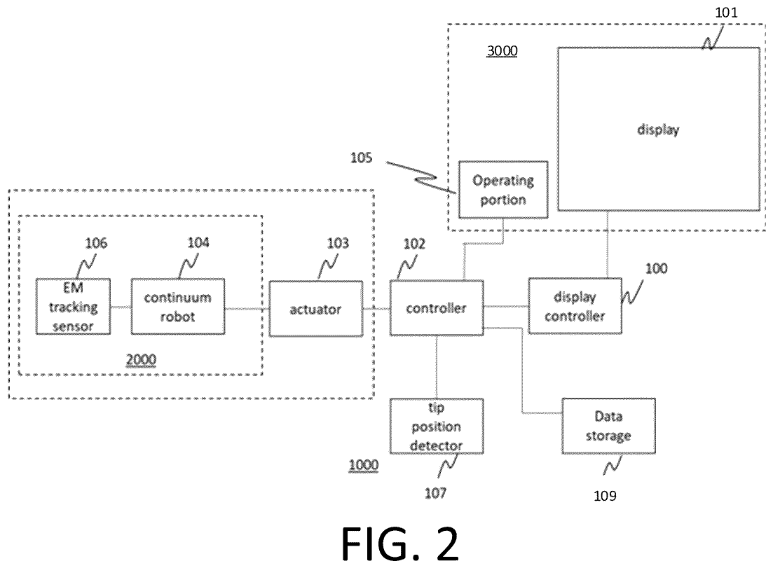

At least one embodiment of a structure of an apparatus or system 1000 is shown in FIGS. 1 to 4C of the present disclosure. As shown in FIGS. 1-4C of the present disclosure, one or more embodiments of a system 1000 for performing imaging and/or visualization (e.g., for a continuum robot, a ureteroscope, a continuum robotic ureteroscope, etc.) may include one or more of the following: a display controller 100, a display 101-1, a display 101-2, a controller 102, an actuator 103, a continuum device 104, an operating portion 105, an EM tracking sensor 106, a catheter tip position detector 107, and a rail 108 (for example, as shown in at least FIGS. 1-2). The system 1000 may include one or more processors, such as, but not limited to, a display controller 100, a controller 102, a CPU 120, a controller 50, a CPU 51, a console or computer 1200 or 1200′, a CPU 1201, any other processor or processors discussed herein, etc., that operate to execute a software program and to control display of a navigation screen on one or more displays 101. The one or more processors (e.g., the display controller 100, the controller 102, the CPU 120, the controller 50, the CPU 51, the console or computer 1200 or 1200′, the CPU 1201, any other processor or processors discussed herein, etc.) may generate a three dimensional (3D) model of a structure (for example, a branching structure like for a kidney of a patient, a urinary system of a patient, an object to be imaged, tissue to be imaged, etc.) based on images, such as, but not limited to, CT images, MRI images, etc. Alternatively, the 3D model may be received by the one or more processors (e.g., the display controller 100, the controller 102, the CPU 120, the controller 50, the CPU 51, the console or computer 1200 or 1200′, the CPU 1201, any other processor or processors discussed herein, etc.) from another device. A two dimensional (2D) model may be used instead of 3D model in one or more embodiments. The 2D or 3D model may be generated before a navigation starts. Alternatively, the 2D or 3D model may be generated in real-time (in parallel with the navigation). In the one or more embodiments discussed herein, examples of generating a model of branching structure and/or a model of a urinary system are explained. However, the models may not be limited to a model of branching structure and/or a model of a urinary system. For example, a model of a route direct to a target may be used instead of the branching structure and/or the urinary system. Alternatively, a model of a broad space may be used, and the model may be a model of a place or a space where an observation or a work is performed by using a continuum robot 104.

While not limited to such a configuration, the display controller 100 may acquire position information of the continuum robot 104 from a controller 102. Alternatively, the display controller 100 may acquire the position information directly from a tip position detector 107. The continuum robot 104 may be a catheter device, a ureteroscope, etc. The continuum robot 104 may be attachable/detachable to the actuator 103, and the continuum robot 104 may be disposable.

In one or more embodiments, the one or more processors, such as the display controller 100, may generate and output a navigation screen to the one or more displays 101-1, 101-2 based on the 3D model and the position information by executing the software. The navigation screen indicates a current position of the continuum robot or endoscope/ureteroscope 104 on the 3D model. By the navigation screen, a user can recognize the current position of the continuum robot or endoscope/ureteroscope 104 in the object, target, or specimen.

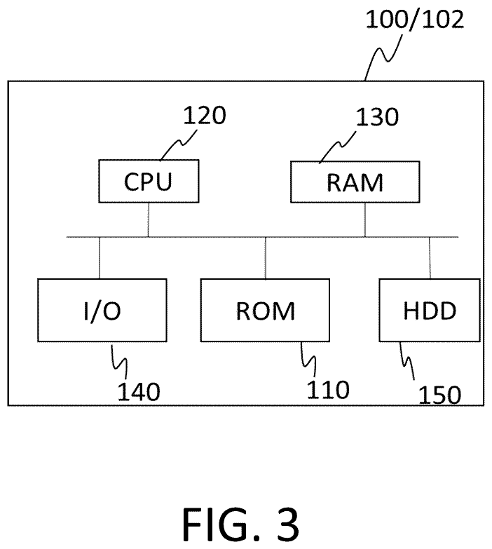

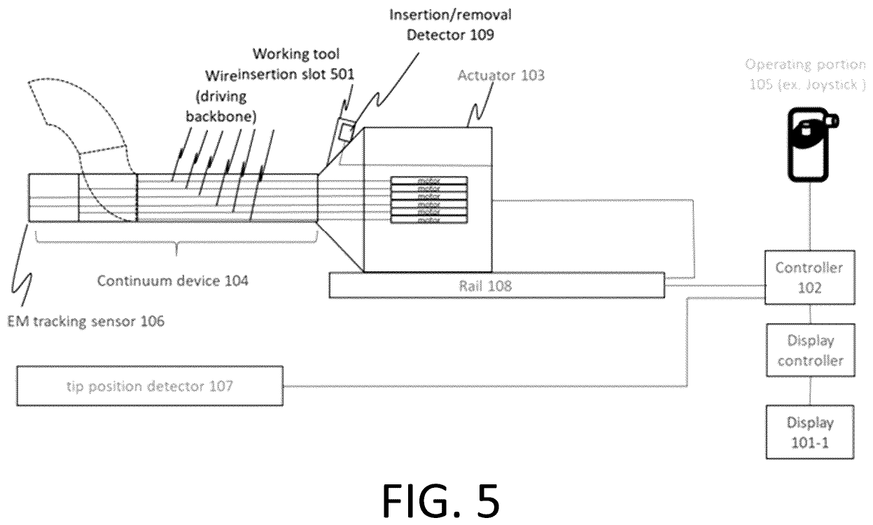

In one or more embodiments, the one or more processors, such as, but not limited to, the display controller 100 and/or the controller 102, may include, as shown in FIG. 3, at least one storage Read Only Memory (ROM) 110, at least one central processing unit (CPU) 120, at least one Random Access Memory (RAM) 130, at least one input and output (I/O) interface 140 and at least one Hard Disc Drive (HDD) 150. A Solid State Drive (SSD) may be used instead of HDD 150. In one or more additional embodiments, the one or more processors, and/or the display controller 100 and/or the controller 102, may include structure as shown in FIGS. 5, 9, 18, and 21-25 as further discussed below.

The ROM 110 and/or HDD 150 operate to store the software in one or more embodiments. The RAM 130 may be used as a work memory. The CPU 120 may execute the software program developed in the RAM 130. The I/O 140 operates to input the positional information to the display controller 100 and to output information for displaying the navigation screen to the one or more displays 101-1, 101-2. In the embodiments below, the navigation screen may be generated by the software program. In one or more other embodiments, the navigation screen may be generated by a firmware.

In one or more embodiments, the data storage 109 (see FIG. 2) operates to store the model (e.g., a segmented kidney model) created from a preoperative CT scan.

In one or more embodiments, the endoscope 104 may be a scope device. The endoscope 104 can be attachable/detachable to the actuator 103 and the endoscope 104 can be disposable.

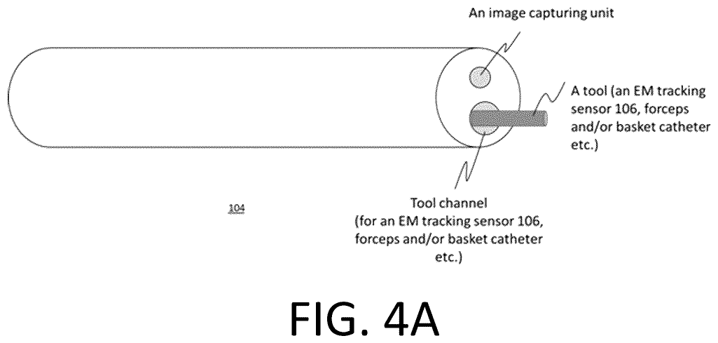

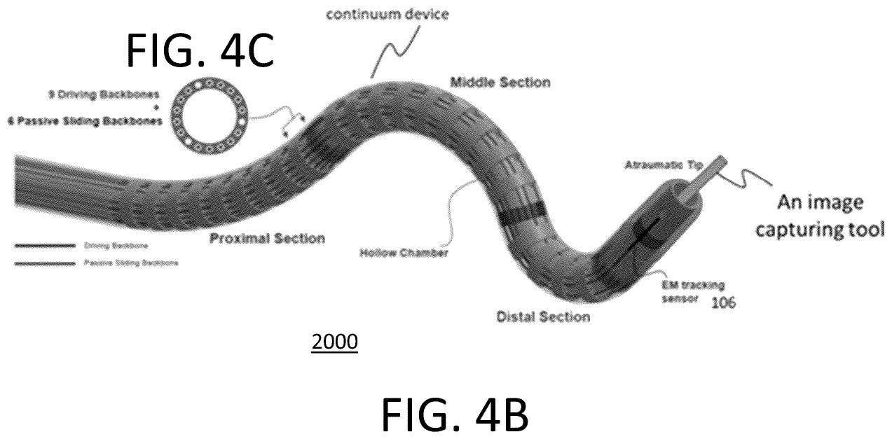

FIGS. 4A-4C show at least one embodiment of a continuum robot or endoscope/ureteroscope 104 that may be used in the system 1000 or any other system discussed herein. In FIG. 4A, the continuum robot or endoscope/ureteroscope 104 may have an image capturing unit or tool and one or more tool channel(s). In the tool channel (e.g., as shown in FIG. 4A), a medical tool, such as, but not limited to, an electro-magnetic (EM) tracking sensor 106, forceps, and/or a basket catheter may be inserted.

As shown in FIG. 4B, the continuum robot or endoscope/ureteroscope 104 may include a continuum device and an image capturing tool inserted in the continuum robot or endoscope/ureteroscope 104. The continuum robot or endoscope/ureteroscope 104 may include a proximal section, a middle section, and a distal section, and each of the sections may be bent by a plurality of driving wires (driving liner members, such as a driving backbone or backbones). In one or more embodiments, the continuum robot may be a catheter device or scope 104. The posture of the catheter device or scope 104 may be supported by supporting wires (supporting liner members, for example, passive sliding backbones). The driving wires may be connected to the actuator 103. The actuator 103 may include one or more motors and drives for each of the sections of the catheter, scope, continuum robot, endoscope, ureteroscope 104 by pushing and/or pulling the driving wires (driving backbones). The actuator 103 may proceed or retreat along a rail 108 (e.g., to translate the actuator 103, the continuum robot/catheter 104, etc.), and the actuator 103 and continuum robot 104 may proceed or retreat in and out of the patient's body or other target, object, or specimen (e.g., tissue, a kidney (e.g., a kidney that has been removed from a body), etc.). As shown in FIG. 4C, the catheter device 104 may include a plurality of driving backbones and may include a plurality of passive sliding backbones. In one or more embodiments, the catheter device 104 may include at least nine (9) driving backbones and at least six (6) passive sliding backbones. The catheter device 104 may include an atraumatic tip at the end of the distal section of the catheter device 104.

One or more embodiments of the catheter/continuum robot or endoscope/ureteroscope 104 may include an electro-magnetic (EM) tracking sensor 106. One or more other embodiments of the catheter/continuum robot 104 may not include or use the EM tracking sensor 106. The electro-magnetic tracking sensor (EM tracking sensor) 106 may be attached to the tip of the continuum robot or endoscope 104/ureteroscope. In this embodiment, a robot 2000 may include the continuum robot 104 and the EM tracking sensor 106 (as seen diagrammatically in FIG. 2), and the robot 2000 may be connected to the actuator 103.

One or more devices or systems, such as the system 1000, may include a tip position detector 107 that operates to detect a position of the EM tracking sensor 106 and to output the detected positional information to the controller 102 (e.g., as shown in FIG. 5).

The controller 102 operates to receive the positional information of the tip of the continuum robot or endoscope/ureteroscope 104 from the tip position detector 107. The controller 102 operates to control the actuator 103 in accordance with the manipulation by a user (e.g., manually), or automatically (e.g., by a method or methods run by one or more processors using software, by the one or more processors, etc.) via one or more operation/operating portions or operational controllers 105 (e.g., such as, but not limited to a joystick as shown in FIG. 5). The one or more displays 101-1, 101-2 and/or operation portion or operational controllers 105 may be used as a user interface 3000 (also referred to as a receiving device) (e.g., as shown diagrammatically in FIG. 2). In an embodiment shown in FIG. 2 and FIG. 5, the system 1000 may include, as an operation unit, the display 101-1 (e.g., such as, but not limited to, a large screen user interface with a touch panel, first user interface unit, etc.), the display 101-2 (e.g., such as, but not limited to, a compact user interface with a touch panel, a second user interface unit, etc.) and the operating portion 105 (e.g., such as, but not limited to, a joystick shaped user interface unit having shift lever/button, a third user interface unit, a gamepad, or other input device, etc.).

The controller 102 may control the continuum robot or endoscope/ureteroscope 104 based on an algorithm known as follow the leader (FTL) algorithm. By applying the FTL algorithm, the middle section and the proximal section (following sections) of the continuum robot or endoscope/ureteroscope 104 may move at a first position in the same way as the distal section moved at the first position or a second position near the first position (e.g., during insertion of the continuum robot/catheter or endoscope/ureteroscope 104). Similarly, the middle section and the distal section of the continuum robot or endoscope/ureteroscope 104 may move at a first position in the same way as the proximal section moved at the first position or a second position near the first position (e.g., during removal of the continuum robot/catheter or endoscope/ureteroscope 104). Alternatively, the continuum robot/catheter or endoscope/ureteroscope 104 may be removed by automatically or manually moving along the same path that the continuum robot/catheter or endoscope/ureteroscope 104 used to enter a target (e.g., a body of a patient, an object, a specimen (e.g., tissue), etc.) using the FTL algorithm.

Any of the one or more processors, such as, but not limited to, the controller 102 and the display controller 100, may be configured separately. As aforementioned, the controller 102 may similarly include a CPU 120, a RAM 130, an I/O 140, a ROM 110, and a HDD 150 as shown diagrammatically in FIG. 3. Alternatively, any of the one or more processors, such as, but not limited to, the controller 102 and the display controller 100, may be configured as one device (for example, the structural attributes of the controller 100 and the controller 102 may be combined into one controller or processor, such as, but not limited to, the one or more other processors discussed herein (e.g., computer, console, or processor 1200, 1200′, etc.).

The system 1000 may include a tool channel for a camera, biopsy tools, or other types of medical tools (as shown in FIG. 5). For example, the tool may be a medical tool, such as an endoscope, ureteroscope, a forceps, a needle or other biopsy tools, etc. In one or more embodiments, the tool may be described as an operation tool or working tool. The working tool may be inserted or removed through a working tool insertion slot 501 (as shown in FIG. 5).

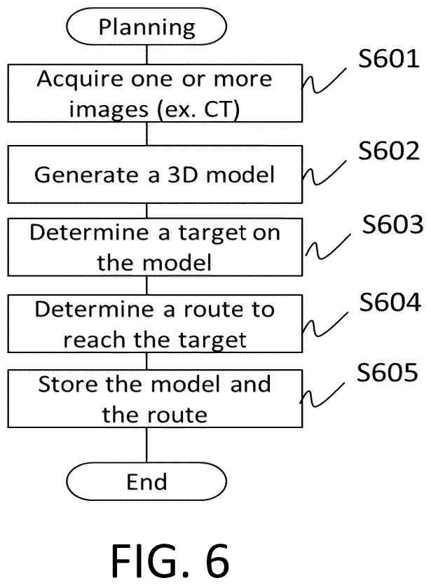

One or more embodiments of the present disclosure may include or use one or more planning methods for planning an operation of the continuum robot or endoscope/ureteroscope 104. At least one embodiment example is shown in FIG. 6 of the present disclosure. The steps of FIG. 6 may be performed by executing a software program read from memory (e.g., the ROM 110, the HDD 150, any other memory discussed herein or known to those skilled in the art, etc.) by a processor, such as, but not limited to, the CPU 120, the processor 1200, the processor 1200′, any other processor or computer discussed herein, etc. In step 601, images such as CT or MRI images are acquired. In step 602, a three dimensional (3D) model of an anatomical structure (for example, a urinary system, a kidney, etc.) is generated based on the acquired images. In step 603, a target in the urinary system is determined based on a user instruction or is determined automatically based on set or predetermined information. In step 604, a route of the endoscope 104 to reach the target in the target object or specimen (e.g., in a urinary system) is determined based on a user instruction. Step 604 may be optional in one or more embodiments. In step 605, the generated three dimensional (3D) model and the decided route on the model are stored in a memory, such as, but not limited to, the RAM 130, the HDD 150, any other memory discussed herein, etc. In this way, a 3D model of the target object or specimen (e.g., a urinary system, a kidney, etc.) is generated and a target and a route on the 3D model is determined and stored before the operation of the endoscope 104 is started.

In one or more of the embodiments discussed below, one or more embodiments of using a manual scope device and a robotic scope device are explained.

One or more embodiments of the present disclosure may be used for post procedure, such as, but not limited to, lithotripsy.

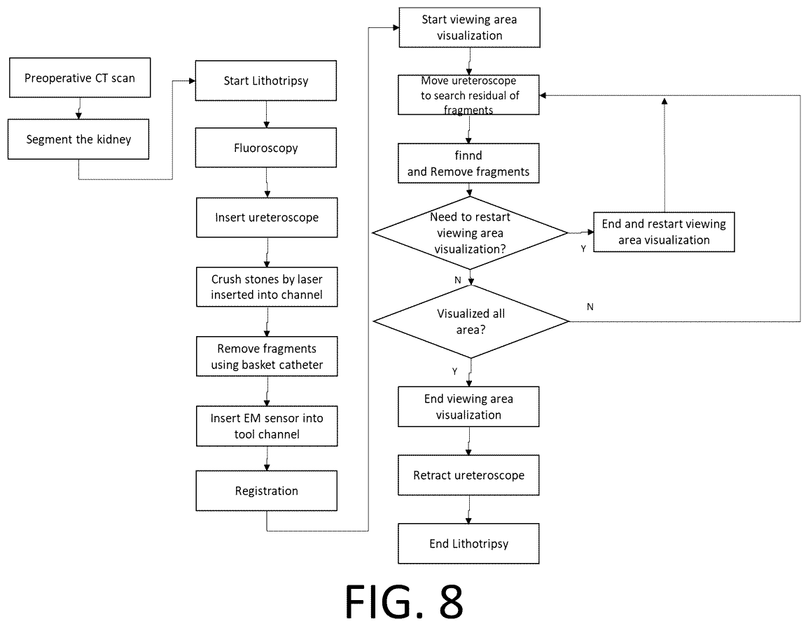

FIGS. 8-10 show a flowchart, a schematic diagram, and views on monitors, respectively of one or more embodiments. Before a lithotripsy procedure, a patient may take a preoperative CT scan to identify a urinary stone, and the inner wall of the kidney is segmented (see two steps in the first column of FIG. 8). The segmented kidney model may be stored in the data storage or sent to one or more processors for use. The preoperative CT scan may be taken on a different day from the lithotripsy procedure.

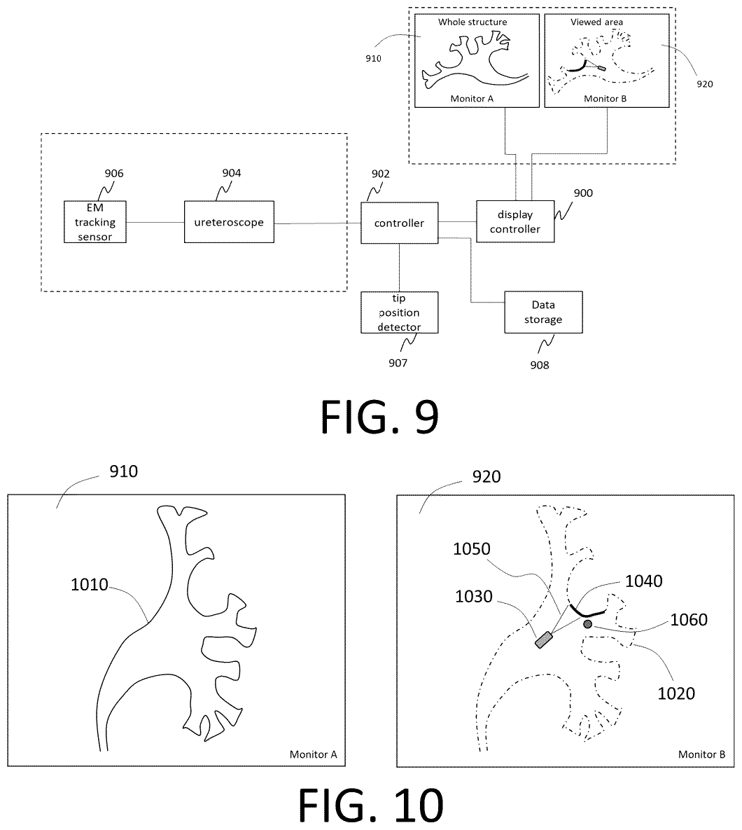

At the beginning of the lithotripsy procedure (see second column of FIG. 8), a physician may take a fluoroscopic image to confirm the location of the urinary stone. Then the physician may insert a ureteroscope (e.g., the ureteroscope 104, the ureteroscope 904 as shown in FIG. 9, etc.)) into the urinary system and may navigate the ureteroscope (e.g., the ureteroscope 104, the ureteroscope 904 as shown in FIG. 9, etc.)) toward the urinary stone shown in the fluoroscopic image. Once the ureteroscope (e.g., the ureteroscope 104, the ureteroscope 904 as shown in FIG. 9, etc.)) reaches the urinary stone, a laser fiber may be inserted through a tool channel of the ureteroscope (e.g., the ureteroscope 104, the ureteroscope 904 as shown in FIG. 9, etc.)), and the physician may crush the stone into fragments (or the ureteroscope (e.g., the ureteroscope 104, the ureteroscope 904 as shown in FIG. 9, etc.)) may operate to crush the stone into fragments automatically in response to reaching the urinary stone in one or more embodiments). The physician may then retract the laser fiber and may deploy a tool, such as a basket catheter, through the tool channel to remove all fragments. These steps may be repeated until the ureteroscope (e.g., the ureteroscope 104, the ureteroscope 904 as shown in FIG. 9, etc.)) and/or the physician removes the fragments as much as possible as the physician and/or the ureteroscope (e.g., the ureteroscope 104, the ureteroscope 904 as shown in FIG. 9, etc.)) can.

One or more embodiments may include a visualization mode, step, method, or technique. For example, after the fragments are removed, an electromagnetic tracking sensor (EM tracking sensor) may be inserted through the tool channel and may be stopped at the tip of the ureteroscope (e.g., the ureteroscope 104, the ureteroscope 904 as shown in FIG. 9, etc.)) to obtain the location and the orientation of the ureteroscope (e.g., the ureteroscope 104, the ureteroscope 904 as shown in FIG. 9, etc.)). In one or more embodiments, positional information may include orientation and/or position/location information. Registration (see e.g., bottom step of second column in FIG. 8), such as, point-set registration, to obtain the transform between the patient coordinate frame and EM tracking system coordinate frame may be performed using landmarks in the target object or specimen (e.g., a kidney) such as the renal pelvis and each calyx in the case of the target object or specimen being the kidney. The ureteroscope (e.g., the ureteroscope 104, the ureteroscope 904 as shown in FIG. 9, etc.)) may also show a virtual First-Person-View onto Monitor A (see e.g., 910 in FIGS. 9-10) after the registration process, which shows the virtual ureteroscopic view corresponding to the actual ureteroscopic view captured in the same location of the kidney.

In one or more embodiments, the parameters of the virtual First-Person-View, such as, but not limited to, Field-Of-View (FOV) and/or focal length, were adjusted to show the corresponding view of the actual ureteroscopic view. The physician may visually check whether the virtual First-Person-View matches, or matches well (e.g., within a certain percentage of accuracy), with the actual ureteroscopic view. If the virtual and actual view did not match due to a registration error and/or an offset between the position of the actual camera and the EM tacking sensor (e.g., the EM tracking sensor 106, the EM tracking sensor 1030, etc.), the ureteroscope (e.g., the ureteroscope 104, the ureteroscope 904 as shown in FIG. 9, etc.)) and/or the physician may adjust the transform obtained from the point-set registration process to match the views (such that the views match, such that the views match within a certain percentage of accuracy, etc.).

In a case where the physician or other practitioner instructs an apparatus or system (e.g., the system of FIG. 9, any other apparatus or system discussed herein), etc.) to start a visualization mode of a viewed area (see top step of third column of FIG. 8), the whole shape of the segmented kidney model stored in the data storage (or sent to a processor, such as the controller 902, the display controller 900, any other processor discussed herein, etc.) and the real time location of the EM tracking sensor (e.g., the EM tracking sensor 106, the EM tracking sensor 1030, etc.) may be displayed in the virtual view in yellow (or in any other predetermined or set color) on Monitor A 910.

On Monitor B 920 (see FIGS. 9-10), a real-time location of the EM tracking sensor 1030, a semi-transparent yellow (a first color, which may be any set or predetermined color (and is not limited to yellow)) shape of the segmented kidney model 1020 and a cone (or any other geometric shape that is set or predetermined (and is not limited to a cone)) shape indicating the Field-Of-View (FOV) 1050 of the ureteroscope 904 are displayed as a virtual view. In one or more embodiments, the shape of the FOV may be based at the tip of the ureteroscope and the angle may be defined by the camera FOV or the FOV of the image capturing tool (e.g., a camera). Alternatively, the cone (or other geometric shape) shape may be narrow to only include a portion of the full FOV (for example 95% in a case where the image may not be as clear at an edge). In one or more embodiments, the area or portion of an image to paint or color may be defined by the intersection or overlap of the cone (or other geometric shape) indicating the FOV and a surface of the 3D image. In one or more embodiments, the area or portion to paint or color may be defined as the intersection only where the image capturing tool is within a specified distance from the surface of the 3D image based on a focal depth of the camera or image capturing tool.

The physician (or other medical practitioner) may move the ureteroscope (e.g., the ureteroscope 104, the ureteroscope 904 as shown in FIG. 9, etc.)) to search for a residual of the fragments of the urinary stone in the urinary system (see second step in third column of FIG. 8). During the search, when the cone shape in the virtual view displayed on the Monitor B hits an area of the semi-transparent shape of the segmented kidney model, the color of the area (an inner surface of the kidney model) changes into a solid red or other color 1040 (a second color which is different from the first color and may be any predetermined or set color (and is not limited to red)).

The color change operates to help the physician (or other medical practitioner) to recognize visually an area of the kidney where the physician (or other medical practitioner) has already searched in the real kidney. The visualization prevents the physician (or other medical practitioner) from overlooking the fragments of the stone and searching the same area again, and the visualization helps shorten the time to search for the residual of the fragments of the urinary stone. In one or more embodiments, a redundant search may be restrained or avoided, and damage to a human body, object, or specimen by the search may be reduced. In the end, a possibility of causing complications is reduced or is avoided.

Once all areas of the semi-transparent shape of the segmented kidney model change into a solid red color (or other set or predetermined color for the second color), a message to indicate the completion of the search may show up, or be displayed, on the Monitor B 920. The physician (or other medical practitioner) may stop the visualization mode of the viewed area, and may retract the ureteroscope (e.g., the ureteroscope 104, the ureteroscope 904 as shown in FIG. 9, etc.)).

As aforementioned, in one or more embodiments, the discussed features for performing imaging, visualization, color change(s), etc. may be used to restrain or avoid overlooking fragments of a urinary stone. Again, in the end, a possibility of causing complications related to additional time performing a procedure or checking an area more than once is reduced or avoided.

In a case where a fragment of the urinary stone is found during the search, the physician may mark the location of the EM tracking sensor (e.g., the EM tracking sensor 106, the EM tracking sensor 1030, etc.) on Monitor B for the future reference 1060 (see marked spot 1060 displayed in/on Monitor B 920 of FIG. 10) and may stop the visualization mode of the viewed area. The EM tracking sensor (e.g., the EM tracking sensor 106, the EM tracking sensor 1030, etc.) may be retracted and the fragment(s) may be removed using a basket catheter deployed through the tool channel as aforementioned. Then EM sensor (e.g., the EM tracking sensor 106, the EM tracking sensor 1030, etc.) may be inserted again, and the physician may restart the visualization mode of the viewed area (see fourth step in the third column of FIG. 8). In a case where visualization does not need to restart and where all area(s) have been visualized or viewed, the visualization mode of the viewed area may be stopped.

In one or more embodiments, the color of the kidney model shown in the virtual view may be changed based on a positional information of the ureteroscope (e.g., the ureteroscope 104, the ureteroscope 904 as shown in FIG. 9, etc.)) in the kidney and the FOV of the ureteroscope (e.g., the ureteroscope 104, the ureteroscope 904 as shown in FIG. 9, etc.)). In one or more embodiments, an EM sensor (e.g., the EM tracking sensor 106, the EM tracking sensor 1030, etc.) may be used to obtain the positional information of the ureteroscope (e.g., the ureteroscope 104, the ureteroscope 904 as shown in FIG. 9, etc.)). Alternatively, a forward kinematics model, a shape-sensing robotic ureteroscope, and/or an image-based localization method may be used to obtain the positional information of the ureteroscope (e.g., the ureteroscope 104, the ureteroscope 904 as shown in FIG. 9, etc.)) as discussed further below.

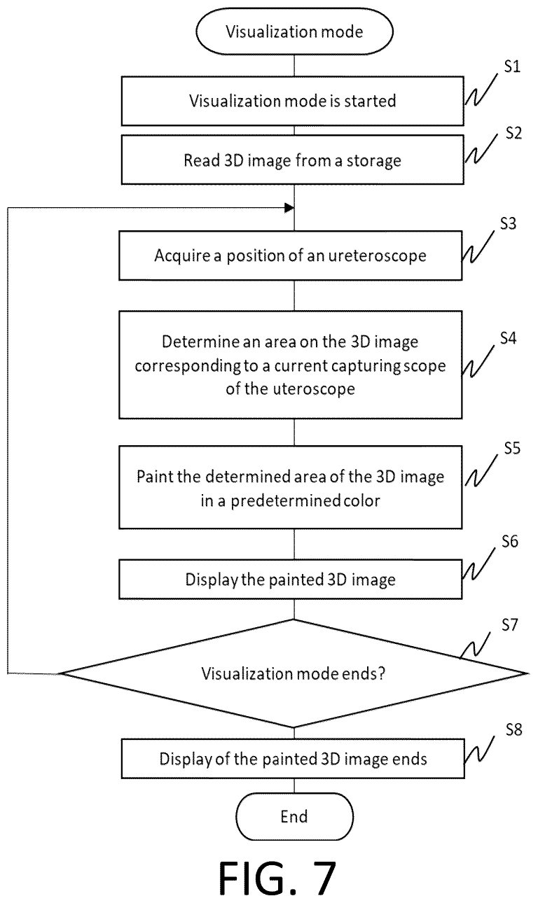

At least one embodiment of a painting 3D model (visualization mode) that may be used is shown in the flowchart of FIG. 7. The steps in the flowchart may be performed by one or more processors (e.g., a CPU, a GPU, a computer 1200, a computer 1200′, any other computer or processor discussed herein, etc.). In S1 of FIG. 7, a visualization mode may be started by a user or may be started automatically by the endoscope (e.g., the ureteroscope 104, the ureteroscope 904 as shown in FIG. 9, etc.)). In S2, the one or more processors operate to read a 3D image of a target, specimen, or object (e.g., an anatomy, a kidney, a tissue, etc.) from a storage (or from an imaging tool in a case where the image is being obtained in real-time or contemporaneously). In S3, the one or more processors operate to acquire a position of a ureteroscope (e.g., the ureteroscope 104, the ureteroscope 904 as shown in FIG. 9, etc.)) from an EM sensor (e.g., the EM tracking sensor 106, the EM tracking sensor 1030, etc.). In S4, the one or more processors operate to determine an area on, or portion of, the 3D image corresponding to a current capturing scope of the ureteroscope (e.g., the ureteroscope 104, the ureteroscope 904 as shown in FIG. 9, etc.)).

In one or more embodiments, a rendering software may determine an area on, or a portion of, a 3D model of an anatomy (for example, the kidney model) that corresponds to an area or portion of the actual anatomy or target specimen or object that is currently captured by the ureteroscope (e.g., the ureteroscope 104, the ureteroscope 904 as shown in FIG. 9, etc.)). The area on the 3D model is determined based on a current position of the ureteroscope and the FOV that the ureteroscope (e.g., the ureteroscope 104, the ureteroscope 904 as shown in FIG. 9, etc.)) can capture. The rendering software renders the 3D image corresponding to a current captured image captured by the ureteroscope (e.g., the ureteroscope 104, the ureteroscope 904 as shown in FIG. 9, etc.)) in one or more embodiments. The rendered 3D image is displayed in the virtual view on the monitor A 910 and/or the monitor B 920.

In S5, the one or more processors operate to perform painting processing to paint the determined area or portion of the 3D image in a predetermined color. In this embodiment, the one or more processors change the color of the area on the 3D model determined by the rendering software from a first color (for example, yellow) to a second color that is different from the first color (for example, red). The color of the first color and the color of the second color are not limited to the respective examples of yellow and red as aforementioned. The first color may be for example, transparent or a color other than yellow. The one or more processors may change the color of an internal surface of the 3D model and/or an outer surface of the 3D model. In one or more embodiments, in a case where the rendering software determines an internal surface of the 3D model as the area or portion captured by the ureteroscope (e.g., the ureteroscope 104, the ureteroscope 904 as shown in FIG. 9, etc.)), colors of both sides of the area (an inner wall of the 3D model and an outer wall of the 3D model) may be changed from the first color to the second color.

In S6, the painted 3D image may be displayed in the virtual view. Once the color of an area or portion of the 3D model is changed, the one or more processors operate to keep the color of the area or portion (or to keep displaying the color of the area or portion on a monitor (e.g., monitor A 910, monitor B 920, any other display or monitor discussed herein, etc.), even in a case where the ureteroscope is moved, until the visualization mode ends.

In S7, the one or more processors operate to determine whether the visualization ends. For example, in a case where a user instructs to end the visualization mode (or in a case where the endoscope (e.g., the ureteroscope 104, the ureteroscope 904 as shown in FIG. 9, etc.)) automatically determines to end the visualization mode), the one or more processors operate to determine that the visualization mode ends and proceeds to S8. In S8, the display of the painted 3D image ends. In a case where the one or more processors determine that the visualization mode is not ended, the one or more processors operate to return to S3.

In one or more embodiments, an example of an operation using a ureteroscope (e.g., the ureteroscope 104, the ureteroscope 904 as shown in FIG. 9, etc.)) is explained as aforementioned. However, one or more features of the present disclosure may be applied to operations using other endoscopes or other imaging devices or systems, such as, but not limited to, a bronchoscope, a vascular endoscope, colonoscope, any other scopes discussed herein or known to those skilled in the art, etc. As such, the present disclosure is not limited to only a ureteroscope.

In one or more embodiments, a slider (or other user-manipulation means or tool) that operates to change the display of the image of the anatomy or of the target, object, or specimen where the painted 3D image is displayed may be used. The slider (or other user-manipulation means or tool) may operate to show the changes in an amount of the paint or color change(s) over a period of time (or a timeframe) of the imaging or procedure.

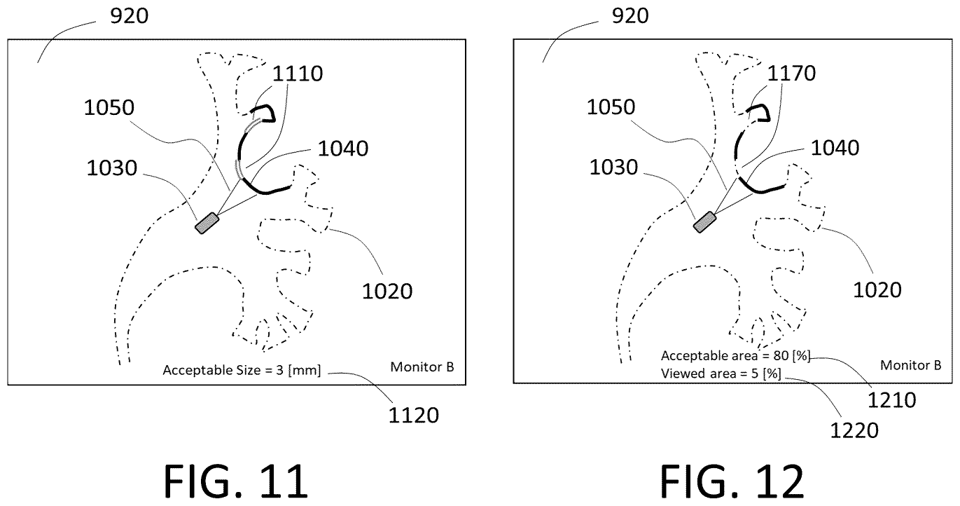

In one or more embodiments, an adjustable criteria may be used, such as, but not limited to, a predetermined or set acceptable size (or a range of sizes) or a threshold for same. In one or more embodiments using a predetermined or set acceptable size (or a range of sizes) or a threshold for same, at least one embodiment example of a display is shown in FIG. 11.

In S4 above, the area of the current capturing scope is determined and displayed. The information may then be used by the physician or an endoscope ((e.g., the ureteroscope 104, the ureteroscope 904 as shown in FIG. 9, etc.) or any other imaging device discussed herein or known to those skilled in the art) to search for the residual of the fragments of the urinary stone and/or to know when enough of the area or portion of an object, target, or specimen that should be viewed has been viewed. The physician or an endoscope ((e.g., the ureteroscope 104, the ureteroscope 904 as shown in FIG. 9, etc.) or any other imaging device discussed herein or known to those skilled in the art) may not need to search for the full or complete area. Even if multiple small areas are not visually checked, the fragments of the urinary stone remaining at those areas are small enough and are likely to be eliminated with urine. To show where the urinary stones or fragments at the unchecked areas are small enough and negligible, a physician may set the acceptable size 1120 of a missed area of the visualization mode of the viewed area before starting the visualization mode of the viewed area (see 1120 in FIG. 11). As the default value, the acceptable size may be set as 3 mm based on the empirical fact that fragments less than 3 mm may be easily eliminated through the urinary tract with urine, causing no complication. That said, physicians or other medical practitioners may change or adjust the set acceptable size 1120 as desired.

After starting the visualization mode of the viewed area, in a case where the cone (or other geometric) shape indicating the Field-Of-View 1050 of the ureteroscope (e.g., the ureteroscope 104, the ureteroscope 904 as shown in FIG. 9, etc.) hits an area of the semi-transparent shape of the segmented kidney model 1020, the color of the area changes into a solid red (or other predetermined or set) color 1040. If the missed area is less than the acceptable size, the color of the area turns into solid pink (or other predetermined or set color) 1110. In a case where the color of all areas of the segmented kidney model changes into solid red or solid pink (or other set colors as desired), a message to indicate the completion of the search shows up on Monitor B 920.

In view of the above, one or more embodiments operate such that the physician (or other medical practitioner) may save time to search for the residuals of the fragments of the urinary stone.

In one or more embodiments, an adjustable criteria may be used, such as, but not limited to, a predetermined or set acceptable percentage or a threshold for same. In one or more embodiments using a predetermined or set acceptable percentage or a threshold for same, at least one embodiment example of a display or monitor is shown in FIG. 12.

A physician may set the acceptable percentage 1210 of completion of the visualization mode of the viewed area before starting the visualization mode of the viewed area.

After starting the visualization mode of the viewed area, in a case where the cone (or other geometric) shape indicating the Field-Of-View of the ureteroscope (e.g., the ureteroscope 104, the ureteroscope 904 as shown in FIG. 9, etc.) hits an area of the semi-transparent shape 1020 of the segmented kidney model, the color of the area changes into a solid red (or other predetermined or set) color. The percentage of the viewed area 1220 is displayed on the monitor B 920. When the red color covers the semi-transparent shape of the segmented kidney model more than the acceptable percentage of completion for the visualization mode of the viewed area, a message to indicate the completion of the search shows up on the monitor (e.g., the monitor B 920).

In view of the above, one or more embodiments operate such that the physician (or other medical practitioner) may save time to search for the residuals of the fragments of the urinary stone.

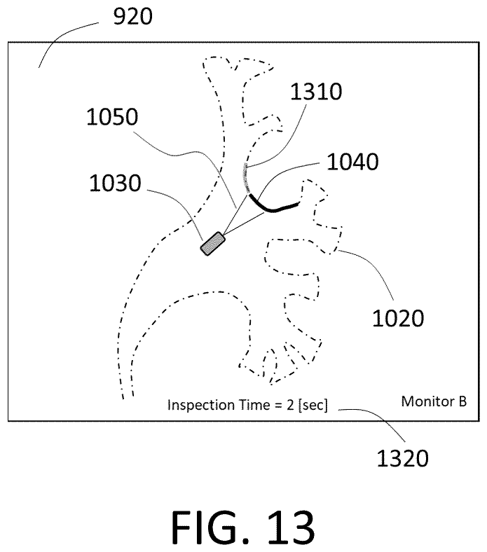

One or more embodiments of the present disclosure employ careful inspection features. For example, FIG. 13 shows at least one embodiment example of a monitor or display using careful inspection features.

A physician (or other medical practitioner) may want to have information as to how carefully the viewed areas were inspected to provide additional information about the process. The carefulness of the inspection may be defined as a length of time that a particular portion of the viewed area was within the Field-of-View in one or more embodiments. A physician (or other medical practitioner) may set the time to define careful inspection 1320 of the viewed area before starting the visualization mode of viewed area.

After starting the visualization mode of the viewed area, in a case where the cone (or other geometric) shape indicating the Field-Of-View of the ureteroscope (e.g., the ureteroscope 104, the ureteroscope 904 as shown in FIG. 9, etc.) hits an area of the semi-transparent shape of the segmented kidney model, the color of the area changes into a semi-transparent red (or other predetermined or set) color 1310, and the opacity of the area changes over time. In a case where the set time indicating careful inspection passes, the color of the area changes into solid red.

In view of the above, one or more embodiments operate such that the physician (or other medical practitioner) may check and/or confirm where a physician (or medical practitioner) carefully checks or inspects based on time.

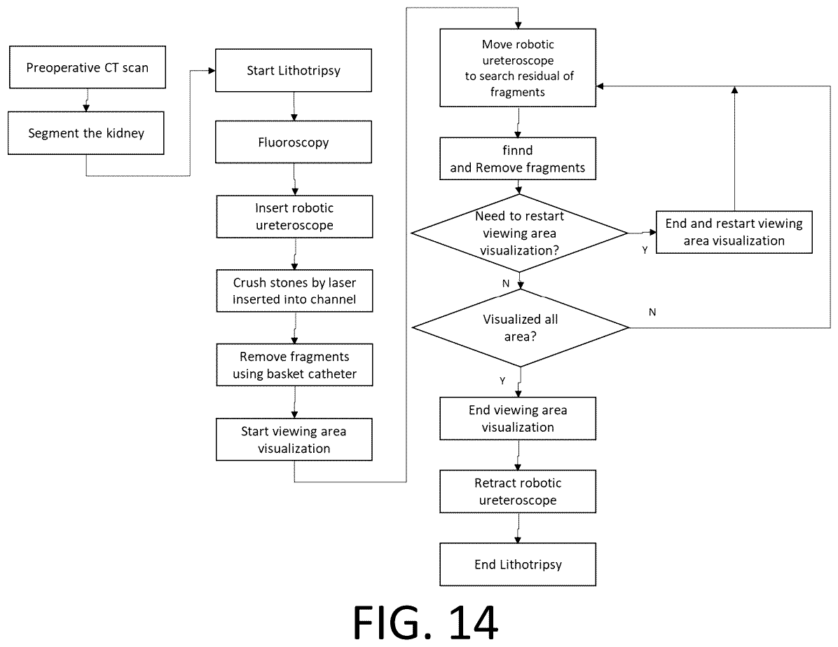

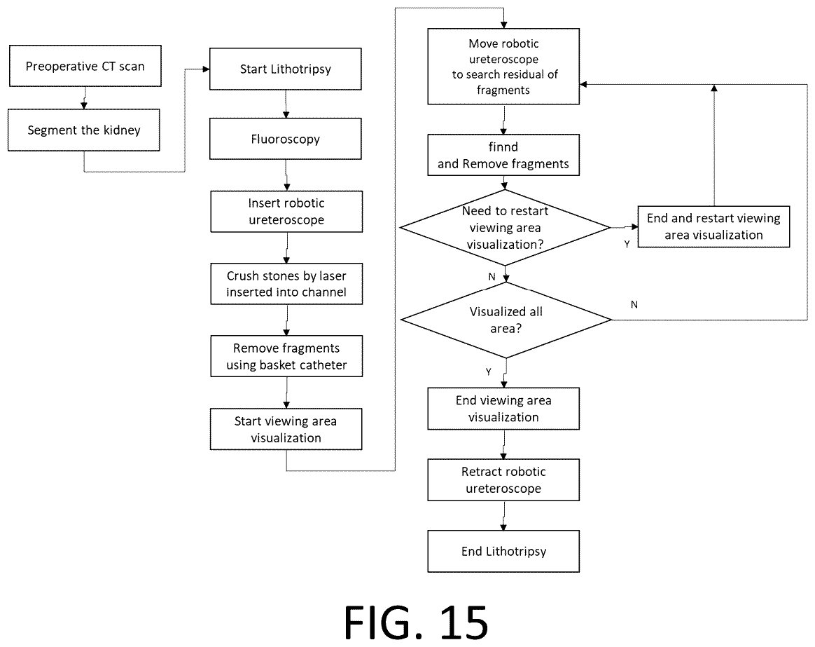

In one or more embodiments, a robotic ureteroscope and forward kinematics may be employed. For example, FIGS. 14, 15, and 16 show a flowchart, a schematic diagram, and views on monitors or displays, respectively, using a robotic ureteroscope and forward kinematics. A physician (or other medical practitioner) may insert a continuum robot as a robotic ureteroscope, and perform lithotripsy. After the physician (or other medical practitioner) removes the fragments of a urinary stone as much as possible as the physician (or other medical practitioner) can, the physician (or other medical practitioner) starts a visualization mode of viewed area. The whole shape of the segmented kidney model stored in the data storage or received from an imaging device, such as a camera or an image capturing tool, may be displayed on Monitor A 1510. On Monitor B 1520, a real-time calculated shape of the robotic ureteroscope 1630, a semi-transparent shape of the segmented kidney model 1620 and a cone shape indicating the Field-Of-View 1650 of the ureteroscope are displayed. As aforementioned, the FOV may be indicated using any geometric shape desired, and is not limited to a cone.

In a case where the physician moves the robotic ureteroscope 1630 to search for a residual of the fragments of the urinary stone, the real-time shape of the robotic ureteroscope 1630 is calculated based on a forward kinematics model. During the search, in a case where the cone (or other geometric) shape hits an area of the semi-transparent shape of the segmented kidney model, the color of the area changes into a solid red (or other set or predetermined) color 1640. Once all areas of the semi-transparent shape of the segmented kidney model change into a solid red (or other set or predetermined) color, a message to indicate the completion of the search shows up on the monitor B 1520. The physician (or other medical practitioner) stops the visualization mode of viewed area, and retracts the ureteroscope 1630.

In view of the above, one or more embodiments operate such that the viewed area may be calculated without using an EM tracking sensor or system.

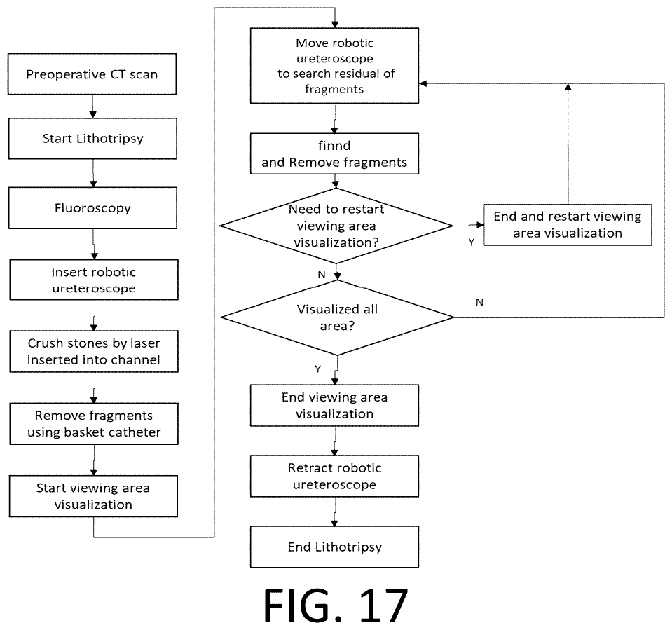

In one or more embodiments, a shape-sensing robotic ureteroscope and image-based depth mapping may be employed. For example, FIGS. 17, 18, and 19 show a flowchart, a schematic diagram, and a view on the monitor or display, respectively, using a shape-sensing robotic ureteroscope and image-based depth mapping. A physician (or other medical practitioner) inserts a continuum robot as a robotic ureteroscope, and performs lithotripsy. After the physician removes the fragments of a urinary stone as much as possible as the physician (or other medical practitioner) can, the physician (or other medical practitioner) starts a visualization mode of viewed area.

The shape of the robotic ureteroscope computed by the shape sensor 1804 and a cone (or other geometric) shape 1950 indicating the Field-Of-View of the ureteroscope are displayed on the monitor 1820. The Image-based depth mapping unit 1810 computes the depth map based on the ureteroscopic view, and displays the inner wall of the kidney 1940 using the computed depth map and the location of the tip of the ureteroscope computed by the shape sensor as a solid part on the monitor 1820. One example of image-based depth mapping method may be found in the following literature: Banach A, King F, Masaki F, Tsukada H, Hata N. Visually Navigated Bronchoscopy using three cycle-Consistent generative adversarial network for depth estimation. Med Image Anal. 2021; 73:102164. doi: 10.1016/j.media.2021.102164, the disclosure of which is incorporated by reference herein in its entirety.

Once the solid part 1940 indicating the inner wall of the kidney covers all of an area of the inner wall of the kidney, a message to indicate the completion of the search shows up on the monitor 1820. The physician (or other medical practitioner) stops the visualization mode of viewed area, and retracts the ureteroscope.

In view of the above, one or more embodiments operate such that the viewed area may be calculated using a camera view of the ureteroscope without using an EM tracking sensor or system.

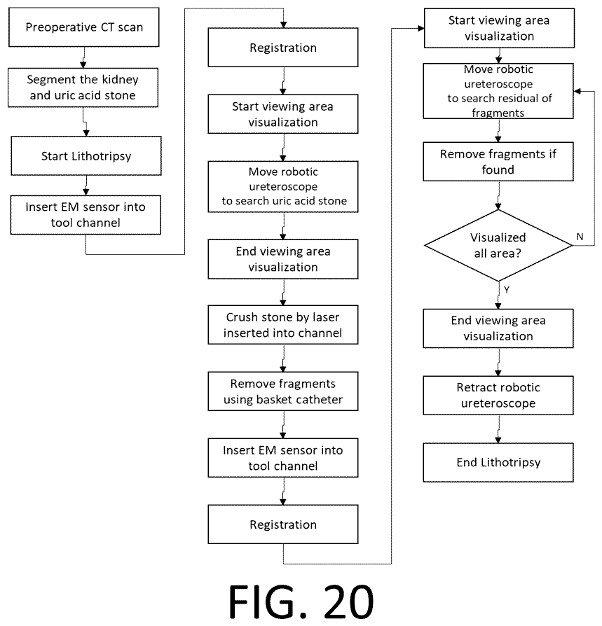

In one or more embodiments, a pre-procedure for a uric acid stone visible by CT and invisible by intraoperative fluoroscopy may be employed. For example, FIG. 20 shows at least one embodiment flowchart example for using a pre-procedure for a uric acid stone.

Before a lithotripsy procedure, a patient may take a preoperative CT scan (or a physician or other medical practitioner may take a CT scan of an object or specimen) to identify a uric acid stone, and the inner model of the kidney and the uric acid stone model may be segmented (see first column of FIG. 20). The segmented kidney model and the uric acid stone may be stored in the data storage or may be received directly from an imaging device, such as a camera or image capturing tool. The preoperative CT scan may be taken on a different day from the lithotripsy procedure.

At the beginning of the lithotripsy procedure for a uric acid stone invisible by intraoperative fluoroscopy, the physician (or other medical practitioner) inserts an EM tracking sensor through the tool channel and stops the EM tracking sensor at the tip of the ureteroscope to obtain the location and the orientation of the camera or image capturing tool of the ureteroscope. In one or more embodiments, positional information may include orientation and/or position/location information. Then, the physician (or other medical practitioner) starts a visualization mode of viewed area. The whole shape of the segmented kidney and the uric acid stone model stored in the data storage (or received from the image device, such as a camera or image capturing tool) are displayed on Monitor A.

On Monitor B, a real-time location of the EM tracking sensor, a semi-transparent yellow (or other predetermined or set color) shape of the segmented kidney model, a semi-transparent green (or other predetermined or set color) shape of the segmented uric acid stone model and a cone (or other geometric) shape indicating the Field-Of-View of the ureteroscope are displayed. The physician (or other medical practitioner) moves the ureteroscope to search for the uric acid stone in the urinary system. During the search, in a case where the cone (or other geometric) shape hits an area of the semi-transparent shape of the segmented kidney model, the color of the area changes into a solid red (or other predetermined or set) color. In a case where the physician (or other medical practitioner) finds the uric acid stone identified by the CT scan, the physician (or other medical practitioner) stops the visualization mode of viewed area, and inserts a laser fiber through a tool channel of the ureteroscope to crush the uric acid stone.

In a case where the physician does not find the uric acid stone and all of the segmented kidney model on Monitor B changes into red, a message indicating that the stone was eliminated through the urinary tract before the procedure shows up on Monitor B.

In view of the above, one or more embodiments operate such that the visualization of viewed area is applied to finding a uric acid stone invisible by intraoperative fluoroscopy, and a physician (or other medical practitioner) may confirm the areas already viewed during a procedure.

In one or more embodiments, an imaging apparatus or system, such as, but not limited to, a robotic ureteroscope, discussed herein may have or include three bendable sections. The visualization technique(s) and methods discussed herein may be used with one or more imaging apparatuses, systems, methods, or storage mediums of U.S. Prov. Pat. App. No. 63/377,983, filed on Sep. 30, 2022, the disclosure of which is incorporated by reference herein in its entirety.

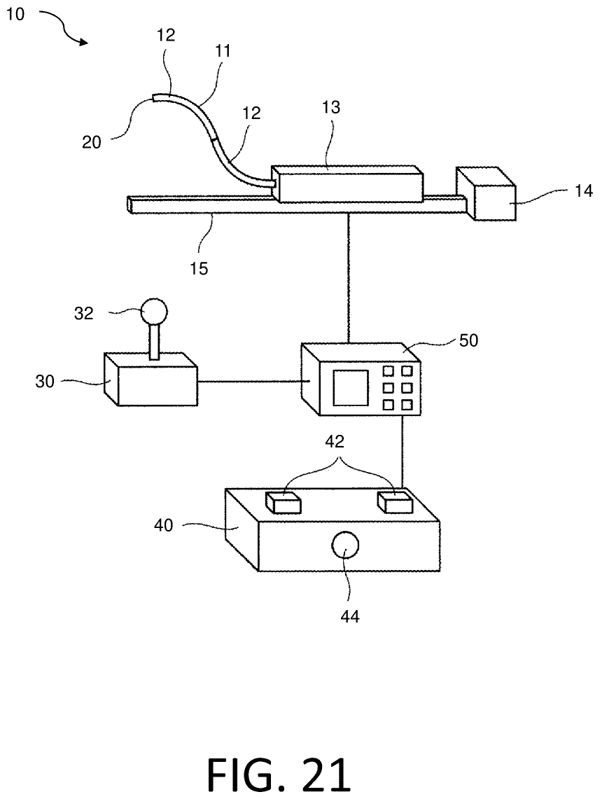

One or more of the aforementioned features may be used with a continuum robot and related features as disclosed in U.S. Provisional Pat. App. No. 63/150,859, filed on Feb. 18, 2021, the disclosure of which is incorporated by reference herein in its entirety. For example, FIGS. 21 to 23 illustrate features of at least one embodiment of a continuum robot apparatus 10 configuration to implement automatic correction of a direction to which a tool channel or a camera moves or is bent in a case where a displayed image is rotated. The continuum robot apparatus 10 enables to keep a correspondence between a direction on a monitor (top, bottom, right or left of the monitor) and a direction the tool channel or the camera moves on the monitor according to a particular directional command (up, down, turn right or turn left) even if the displayed image is rotated.

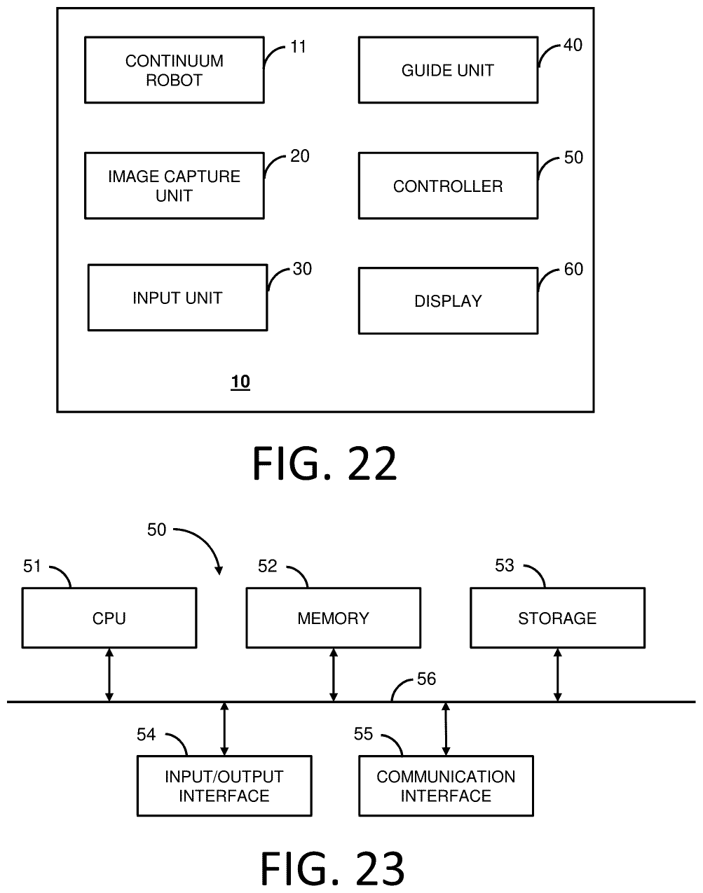

As shown in FIGS. 21 and 22, the continuum robot apparatus 10 may include one or more of a continuum robot 11, an image capture unit 20, an input unit 30, a guide unit 40, a controller 50, and a display 60. The image capture unit 20 can be a camera or other image capturing device. The continuum robot 11 can include one or more flexible portions 12 connected together and configured so they can be curved or rotated about in different directions. The continuum robot 11 can include a drive unit 13, a movement drive unit 14, and a linear drive 15. The movement drive unit 14 causes the drive unit 13 to move along the linear guide 15.

The input unit 30 has an input element 32 and is configured to allow a user to positionally adjust the flexible portions 12 of the continuum robot 11. The input unit 30 may be configured as a mouse, a keyboard, joystick, lever, or another shape to facilitate user interaction. The user may provide an operation input through the input element 32, and the continuum robot apparatus 10 may receive information of the input element 32 and one or more input/output devices, which may include a receiver, a transmitter, a speaker, a display, an imaging sensor, or the like, a user input device, which may include a keyboard, a keypad, a mouse, a position tracked stylus, a position tracked probe, a foot switch, a microphone, or the like. The guide unit 40 is a device that includes one or more buttons, knobs, switches, or the like 42, 44, that a user can use to adjust various parameters the continuum robot 10, such as the speed or other parameters.

FIG. 23 illustrates the controller 50 that may be used in one or more embodiments according to one or more aspects of the present disclosure. The controller 50 is configured to control the elements of the continuum robot apparatus 10 and has one or more of a CPU 51, a memory 52, a storage 53, an input and output (I/O) interface 54, and communication interface 55. The continuum robot apparatus 10 can be interconnected with medical instruments or a variety of other devices, and can be controlled independently, externally, or remotely by the controller 50.

The memory 52 may be used as a work memory. The storage 53 stores software or computer instructions. The CPU 51, which may include one or more processors, circuitry, or a combination thereof, executes the software developed in the memory 52. The I/O interface 54 inputs information from the continuum robot apparatus 10 to the controller 50 and outputs information for displaying to the display 60.

The communication interface 55 may be configured as a circuit or other device for communicating with components included the apparatus 10, and with various external apparatuses connected to the apparatus via a network. For example, the communication interface 55 may store information to be output in a transfer packet and output the transfer packet to an external apparatus via the network by communication technology such as Transmission Control Protocol/Internet Protocol (TCP/IP). The apparatus may include a plurality of communication circuits according to a desired communication form.

The controller 50 may be communicatively interconnected or interfaced with one or more external devices including, for example, one or more data storages, one or more external user input/output devices, or the like. The controller 50 may interface with other elements including, for example, one or more of an external storage, a display, a keyboard, a mouse, a sensor, a microphone, a speaker, a projector, a scanner, a display, an illumination device, or the like.

The display 60 may be a display device configured, for example, as a monitor, an LCD (liquid panel display), an LED display, an OLED (organic LED) display, a plasma display, an organic electro luminescence panel, or the like. Based on the control of the apparatus, a screen may be displayed on the display 60 showing one or more images being captured, captured images, captured moving images recorded on the storage unit, or the like.

The components may be connected together by a bus 56 so that the components can communicate with each other. The bus 56 transmits and receives data between these pieces of hardware connected together, or transmits a command from the CPU 51 to the other pieces of hardware. The components can be implemented by one or more physical devices that may be coupled to the CPU 51 through a communication channel. For example, the controller 50 can be implemented using circuitry in the form of ASIC (application specific integrated circuits) or the like. Alternatively, the controller 50 can be implemented as a combination of hardware and software, where the software is loaded into a processor from a memory or over a network connection. Functionality of the controller 50 can be stored on a storage medium, which may include RAM (random-access memory), magnetic or optical drive, diskette, cloud storage, or the like.

The units described throughout the present disclosure are exemplary and/or preferable modules for implementing processes described in the present disclosure. The term “unit”, as used herein, may generally refer to firmware, software, hardware, or other component, such as circuitry or the like, or any combination thereof, that is used to effectuate a purpose. The modules may be hardware units (such as circuitry, firmware, a field programmable gate array, a digital signal processor, an application specific integrated circuit or the like) and/or software modules (such as a computer readable program or the like) in one or more embodiments. The modules for implementing the various steps are not described exhaustively above. However, where there is a step of performing a certain process, there may be a corresponding functional module or unit (implemented by hardware and/or software) for implementing the same process. Technical solutions by all combinations of steps described and units corresponding to these steps are included in the present disclosure.

While one or more features of the present disclosure have been described with reference to one or more embodiments, it is to be understood that the present disclosure is not limited to the disclosed one or more embodiments. The scope of the claims is to be accorded the broadest interpretation so as to encompass all modifications, equivalent structures, and functions.

A computer, such as the console or computer 1200, 1200′, may perform any of the steps, processes, and/or techniques discussed herein for any apparatus and/or system being manufactured or used, any of the embodiments shown in FIGS. 1-25, any other apparatus or system discussed herein, etc.

There are many ways to control a continuum robot, perform imaging or visualization, or perform any other measurement or process discussed herein, to perform continuum robot method(s) or algorithm(s), and/or to control at least one continuum robot device/apparatus, system and/or storage medium, digital as well as analog. In at least one embodiment, a computer, such as the console or computer 1200, 1200′, may be dedicated to control and/or use continuum robot devices, systems, methods, and/or storage mediums for use therewith described herein.

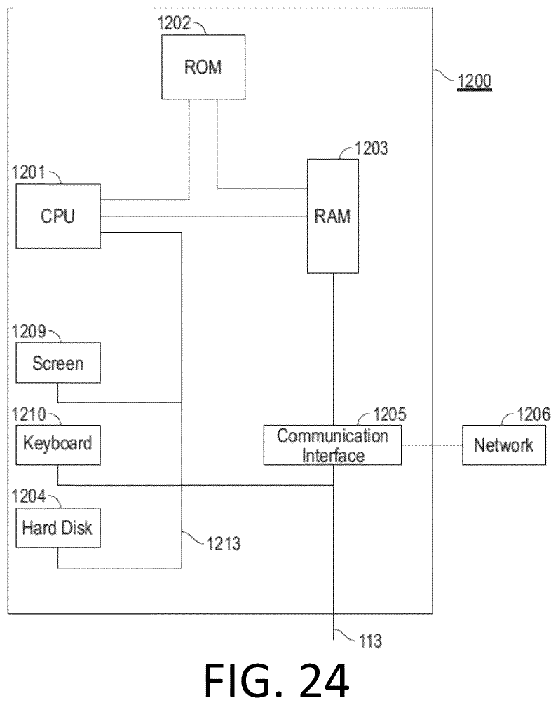

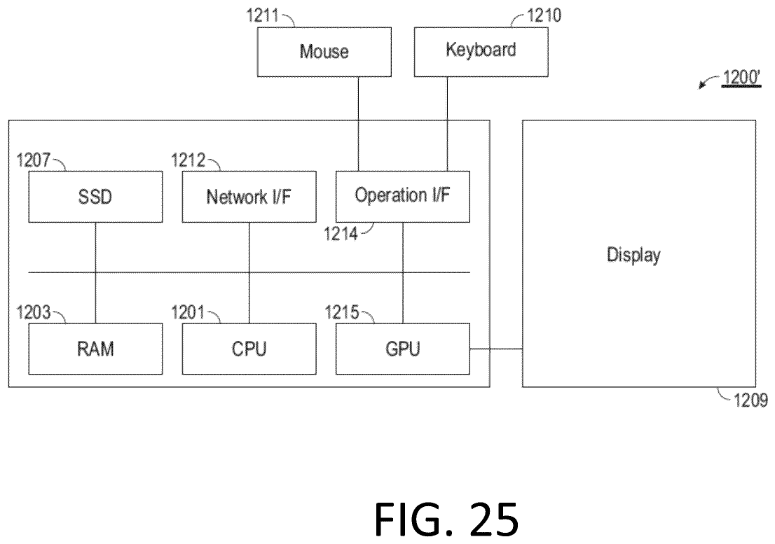

The one or more detectors, sensors, cameras, or other components of the apparatus or system embodiments (e.g. of the system 1000 of FIG. 1 or any other system discussed herein) may transmit the digital or analog signals to a processor or a computer such as, but not limited to, an image processor or display controller 100, a controller 102, a CPU 120, a controller 50, a CPU 51, a display controller 900, a controller 902, a display controller 1500, a controller 1502, a processor or computer 1200, 1200′ (see e.g., at least FIGS. 1-5, 9, 18, and 21-25), a combination thereof, etc. The image processor may be a dedicated image processor or a general purpose processor that is configured to process images. In at least one embodiment, the computer 1200, 1200′ may be used in place of, or in addition to, the image processor or display controller 100 and/or the controller 102 (or any other processor or controller discussed herein, such as, but not limited to, the controller 50, the CPU 51, the display controller 900, the controller 902, the display controller 1500, the controller 1502, etc.). In an alternative embodiment, the image processor may include an ADC and receive analog signals from the one or more detectors or sensors of the system 1000 (or any other system discussed herein). The image processor may include one or more of a CPU, DSP, FPGA, ASIC, or some other processing circuitry. The image processor may include memory for storing image, data, and instructions. The image processor may generate one or more images based on the information provided by the one or more detectors, sensors, or cameras. A computer or processor discussed herein, such as, but not limited to, a processor of the devices, apparatuses or systems of FIGS. 1-5, 9, 18, and 21-25, the computer 1200, the computer 1200′, the image processor, etc. may also include one or more components further discussed herein below (see e.g., FIGS. 24-25).

Electrical analog signals obtained from the output of the system 1000 or the components thereof, and/or from the devices, apparatuses, or systems of FIGS. 1-5, 9, 18, and 21-25, may be converted to digital signals to be analyzed with a computer, such as, but not limited to, the computers or controllers 100, 102 of FIG. 1, the computer 1200, 1200′, any other computer, processor, or controller discussed herein, etc.

As aforementioned, there are many ways to control a continuum robot, correct or adjust an image, or perform any other measurement or process discussed herein, to perform continuum robot method(s) or algorithm(s), and/or to control at least one continuum robot device/apparatus, system and/or storage medium, digital as well as analog. By way of a further example, in at least one embodiment, a computer, such as the computer or controllers 100, 102 of FIG. 1, the console or computer 1200, 1200′, etc., may be dedicated to the control and the monitoring of the continuum robot devices, systems, methods and/or storage mediums described herein.

The electric signals used for imaging may be sent to one or more processors, such as, but not limited to, the processors or controllers 100, 102 of FIGS. 1-5, a computer 1200 (see e.g., FIG. 24), a computer 1200′ (see e.g., FIG. 25), etc. as discussed further below, via cable(s) or wire(s), such as, but not limited to, the cable(s) or wire(s) 113 (see FIG. 24). Additionally or alternatively, the computers or processors discussed herein are interchangeable, and may operate to perform any of the feature(s) and method(s) discussed herein.

Various components of a computer system 1200 (see e.g., the console or computer 1200 as may be used as one embodiment example of the computer, processor, or controllers 100, 102 shown in FIG. 1) are provided in FIG. 24. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., as shown in FIG. 24). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a continuum robot device or system using same, such as, but not limited to, the system 1000, any of the devices/systems of FIGS. 1-5, 9, 18, and 21-25, discussed herein above, via one or more lines 1213), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The computer system 1200 may include one or more additional processors in addition to CPU 1201, and such processors, including the CPU 1201, may be used for controlling and/or manufacturing a device, system or storage medium for use with same or for use with any continuum robot technique(s), and/or use with image correction or adjustment technique(s) discussed herein. The system 1200 may further include one or more processors connected via a network connection (e.g., via network 1206). The CPU 1201 and any additional processor being used by the system 1200 may be located in the same telecom network or in different telecom networks (e.g., performing, manufacturing, controlling, calculation, and/or using technique(s) may be controlled remotely).

The I/O or communication interface 1205 provides communication interfaces to input and output devices, which may include the one or more of the aforementioned components of any of the systems discussed herein (e.g., the controller 100, the controller 102, the displays 101-1, 101-2, the actuator 103, the continuum device 104, the operating portion or controller 105, the EM tracking sensor 106, the position detector 107, the rail 108, etc.), a microphone, a communication cable and a network (either wired or wireless), a keyboard 1210, a mouse (see e.g., the mouse 1211 as shown in FIG. 24), a touch screen or screen 1209, a light pen and so on. The communication interface of the computer 1200 may connect to other components discussed herein via line 113 (as diagrammatically shown in FIG. 25). The Monitor interface or screen 1209 provides communication interfaces thereto.

Any methods and/or data of the present disclosure, such as, but not limited to, the methods for using and/or controlling a continuum robot or catheter device, system, or storage medium for use with same and/or method(s) for imaging and/or visualization, performing tissue or sample characterization or analysis, performing diagnosis, planning and/or examination, controlling a continuum robot device or system, and/or for performing image correction or adjustment technique(s), as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 25), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

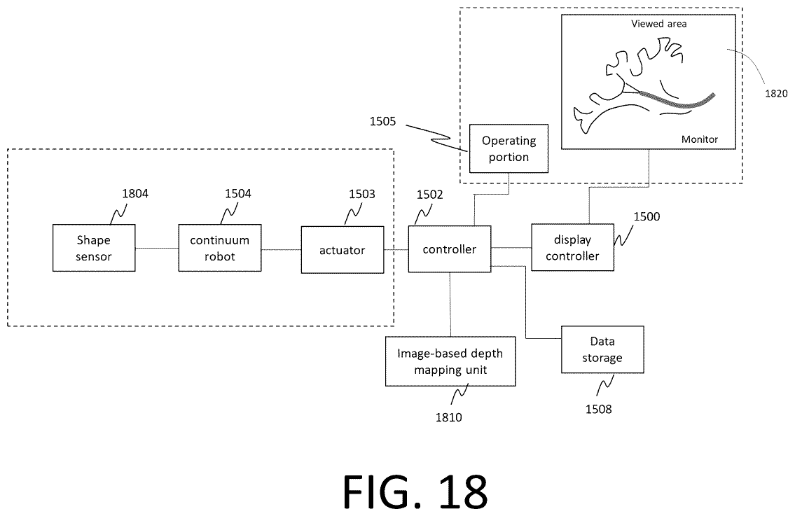

In accordance with at least one aspect of the present disclosure, the methods, devices, systems, and computer-readable storage mediums related to the processors, such as, but not limited to, the processor of the aforementioned computer 1200, the processor of computer 1200′, the controller 100, the controller 102, any of the other controller(s) discussed herein, etc., as described above may be achieved utilizing suitable hardware, such as that illustrated in the figures. Functionality of one or more aspects of the present disclosure may be achieved utilizing suitable hardware, such as that illustrated in FIG. 24. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1201 (as shown in FIG. 24 or FIG. 25, and/or which may be included in the computer, processor, controller and/or CPU 120 of FIGS. 1-5), the controller 902 and/or the display controller 900 (see FIG. 9), the display controller 1500 and/or the controller 1502 (see FIG. 18), CPU 51, the CPU 120, and/or any other controller, processor, or computer discussed herein may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 100, 102, 120, 50, 51, 900, 902, 1500, 1502, 1200, 1200′, any other computer(s) or processor(s) discussed herein, etc.) may include the aforementioned CPU structure, or may be connected to such CPU structure for communication therewith.

As aforementioned, hardware structure of an alternative embodiment of a computer or console 1200′ is shown in FIG. 25. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid-state drive (SSD) 1207. Preferably, the computer or console 1200′ includes a display 1209 (and/or the displays 101-1, 101-2, and/or any other display discussed herein). The computer 1200′ may connect with one or more components of a system (e.g., the systems/apparatuses of FIGS. 1-5, 9, 18, and 21-25, etc.) via the operation interface 1214 or the network interface 1212. The operation interface 1214 is connected with an operation unit such as a mouse device 1211, a keyboard 1210 or a touch panel device. The computer 1200′ may include two or more of each component. Alternatively, the CPU 1201 or the GPU 1215 may be replaced by the field-programmable gate array (FPGA), the application-specific integrated circuit (ASIC) or other processing unit depending on the design of a computer, such as the computer 1200, the computer 1200′, etc.

At least one computer program is stored in the SSD 1207, and the CPU 1201 loads the at least one program onto the RAM 1203, and executes the instructions in the at least one program to perform one or more processes described herein, as well as the basic input, output, calculation, memory writing, and memory reading processes.

The computer, such as the computer 1200, 1200′, the computer, processors, and/or controllers of FIGS. 1-5, 9, 18, and 21-25, any other computer/processor/controller discussed herein, etc., communicates with the one or more components of the apparatuses/systems of FIGS. 1-5, 9, 18, and 21-25, and/or of any other system(s) discussed herein, to perform imaging, and reconstructs an image from the acquired intensity data. The monitor or display 1209 displays the reconstructed image, and may display other information about the imaging condition or about an object to be imaged. The monitor 1209 also provides a graphical user interface for a user to operate a system, for example when performing CT, MRI, or other imaging technique(s), including, but not limited to, controlling continuum robot devices/systems, performing imaging and/or visualization, and/or performing image correction or adjustment technique(s). An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs the system (e.g., the system 1000, the systems/apparatuses of FIGS. 1-5, 9, 18, and 21-25, any other system/apparatus discussed herein, etc.) to start or end the imaging, and/or to start or end continuum robot control(s), and/or performance of imaging and/or visualization technique(s), lithotripsy and/or ureteroscopy methods, and/or image correction or adjustment technique(s). The camera or imaging device as aforementioned may have interfaces to communicate with the computers 1200, 1200′ to send and receive the status information and the control signals.

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Provisional Pat. App. No. 63/150,859, filed on Feb. 18, 2021, the disclosure of which is incorporated by reference herein in its entirety. Such endoscope devices, systems, methods, and/or storage mediums are disclosed in at least: U.S. patent application Ser. No. 17/565,319, filed on Dec. 29, 2021, the disclosure of which is incorporated by reference herein in its entirety; U.S. Pat. App. No. 63/132,320, filed on Dec. 30, 2020, the disclosure of which is incorporated by reference herein in its entirety; U.S. patent application Ser. No. 17/564,534, filed on Dec. 29, 2021, the disclosure of which is incorporated by reference herein in its entirety; and U.S. Pat. App. No. 63/131,485, filed Dec. 29, 2020, the disclosure of which is incorporated by reference herein in its entirety.

Although one or more features of the present disclosure herein have been described with reference to particular embodiments, it is to be understood that these embodiments are merely illustrative of the principles and applications of the present disclosure (and are not limited thereto), and the invention is not limited to the disclosed embodiments. It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications, equivalent structures, and functions.

1. An information processing apparatus comprising:
one or more processors that operate to:
obtain a three dimensional image of an object, target, or sample;
acquire positional information of an image capturing tool inserted in or into the object, target, or sample;
determine, based on the positional information of the image capturing tool and the relative position of the image capturing tool to the object, target, or sample, portions of the object, target, or sample that are a first portion and a second portion, where the first portion corresponds to a portion of the object, target, or sample where the image capturing tool has captured or inspected and the second portion corresponds to a portion of the object, target, or sample where the image capturing tool still is or remains to be captured or to be inspected such that the second portion is uncaptured or uninspected; and
display, based on the acquired positional information, the first portion of the three dimensional image of the object, target, or sample with a first expression which is different from a second expression of the second portion of the three dimensional image.

2. The information processing apparatus of claim 1, wherein the one or more processors further operate to display the second portion and/or the second expression for the second portion along with the first portion and the first expression.

3. The information processing apparatus of claim 1, wherein one or more of the following: (i) the object, target, or sample is one or more of the following: an anatomy, a kidney, a urinary system, or a portion of a kidney or urinary system; and/or (ii) CT data is obtained and/or used to segment the object, target, or sample.

4. The information processing apparatus of claim 1, wherein:
the first portion corresponds to a portion that the image capturing tool has captured or inspected in a Field-of-View of the image capturing tool, and
the second portion corresponds to a portion that remains to be captured or inspected in the Field-of-View of the image capturing tool.

5. The information processing apparatus of claim 1, wherein the captured or inspected first portion represents or corresponds to an overlap between an imaging Field-of-View (FOV) or a view model of the image capturing tool and the surfaces of the object, target, or sample being imaged.

6. The information processing apparatus of claim 5, wherein the view model is a cone or other geometric shape being used for the model, and the portion that the image capturing tool has captured or inspected includes inner surfaces of a kidney that are located within the FOV or the view model.

7. The information processing apparatus of claim 1, wherein the one or more processors further operate to:
receive a predetermined or set acceptable size, or a size within a predetermined or set range, of a missed or uninspected/uncaptured area, the missed or uninspected/uncaptured area being an area or portion that is not captured or inspected, or remains to be captured or inspected, by the image capturing tool; and
display a third portion of the three dimensional image of the object, sample, or target with a third expression which is different from both of the expressions of the first portion and the second portion of the three dimensional image, wherein the third portion corresponds to the missed or uninspected/uncaptured area of which size is equal to or less than the predetermined or set acceptable size.

8. The information processing apparatus of claim 1, wherein the one or more processors further operate to:
receive a predetermined or set acceptable percentage of a completion of a capturing or inspection of the object, target, or sample; and
indicate a completion of the capturing or inspection of the object, target, or sample, in a case where the percentage of an area or portion captured or inspected by the image capturing tool is equal to or more than the predetermined or set acceptable percentage.

9. The information processing apparatus of claim 1, wherein the one or more processors further operate to:
store time information corresponding to a length of time that a particular portion or area is within the Field-of-View of the image capturing tool; and
display the three dimensional image of the anatomy with the first expression of the first portion after the image capturing tool has captured or inspected the first portion for a period of time indicated by the stored time information.

10. The information processing apparatus of claim 9, wherein the stored time information is the accumulated during to have overlap between an imaging Field-of-View of the image capturing tool and the surfaces of the object, target, or sample being imaged.

11. The information processing apparatus of claim 1, wherein the one or more processors further operate to: acquire the positional information of the image capturing tool based on a shape of the image capturing tool calculated based on a forward kinematics model, the positional information including orientation and position or location information.

12. The information processing apparatus of claim 1, wherein the one or more processors further operate to: acquire the positional information of the image capturing tool based on a shape sensor of the image capturing tool, the positional information including orientation and position or location information.

13. The information processing apparatus of claim 1, wherein the one or more processors further operate to: acquire the positional information of the image capturing tool based on a positional information detected by an electromagnetic sensor, the positional information including orientation and position or location information.

14. The information processing apparatus of claim 1, wherein the one or more processors further operate to: display, based on a depth map from or based on a captured image captured by the image capturing tool, the three dimensional image of the object, sample, or target.

15. The information processing apparatus of claim 1, wherein the one or more processors further operate to: display, based on the acquired positional information, the first portion of the three dimensional image of the object, sample, or target with a first color being different from a second color of or used for the second portion of the three dimensional image.

16. A method for imaging comprising:
obtaining a three dimensional image of an object, target, or sample;
acquiring positional information of an image capturing tool inserted in or into the object, target, or sample;
determining, based on the positional information of the image capturing tool and the relative position of the image capturing tool to the object, target, or sample, portions of the object, target, or sample that are a first portion and a second portion, where the first portion corresponds to a portion of the object, target, or sample where the image capturing tool has captured or inspected and the second portion corresponds to a portion of the object, target, or sample where the image capturing tool still is or remains to be captured or to be inspected such that the second portion is uncaptured or uninspected; and
displaying, based on the acquired positional information, the first portion of the three dimensional image of the object, target, or sample with a first expression which is different from a second expression of the second portion of the three dimensional image.

17. The method of claim 16, further comprising: displaying the second portion and/or the second expression for the second portion along with the first portion and the first expression.

18. The method of claim 16, wherein the object, target, or sample may be one or more of the following: an anatomy, a kidney, a urinary system, or a portion of a kidney or urinary system.

19. The method of claim 16, wherein:
the first portion corresponds to a portion that the image capturing tool has captured or inspected in a Field-of-View of the image capturing tool, and
the second portion corresponds to a portion that remains to be captured or inspected in the Field-of-View of the image capturing tool.

20. The method of claim 16, wherein the displaying of the three dimensional image of the object, sample, or target is based on a depth map from or based on a captured image captured by the image capturing tool.

21. The method of claim 16, further comprising displaying, based on the acquired positional information, the first portion of the three dimensional image of the object, sample, or target with a first color being different from a second color of or used for the second portion of the three dimensional image.

22. A non-transitory storage medium storing at least one program to be executed by a processor to perform a method for imaging, where the method comprises:
obtaining a three dimensional image of an object, target, or sample;
acquiring positional information of an image capturing tool inserted in or into the object, target, or sample;
determining, based on the positional information of the image capturing tool and the relative position of the image capturing tool to the object, target, or sample, portions of the object, target, or sample that are a first portion and a second portion, where the first portion corresponds to a portion of the object, target, or sample where the image capturing tool has captured or inspected and the second portion corresponds to a portion of the object, target, or sample where the image capturing tool still is or remains to be captured or to be inspected such that the second portion is uncaptured or uninspected; and
displaying, based on the acquired positional information, the first portion of the three dimensional image of the object, target, or sample with a first expression which is different from a second expression of the second portion of the three dimensional image.

23. A method for performing lithotripsy comprising:
obtaining a Computed Tomography (CT) scan;
segmenting an object, target, or sample;
starting lithotripsy;
inserting an Electro Magnetic (EM) sensor into a tool channel, or disposing the EM sensor at a tip of a catheter or probe of a robotic or manual ureteroscope or an imaging apparatus or system;
performing registration;
starting visualization of a viewing area;
moving the imaging apparatus or system or the robotic or manual ureteroscope to search for a urinary stone in an object or specimen;
crushing the urinary stone into fragments using a laser inserted into the tool channel;
removing the fragments of the urinary stone using a basket catheter;
inserting the EM sensor into the tool channel, or disposing the EM sensor at the tip of the catheter or probe of the robotic or manual ureteroscope or the imaging apparatus or system;
performing registration;
starting visualization of a viewing area;
displaying a first portion and a second portion of the viewing area, where the first portion indicates a portion that has been captured or inspected and the second portion indicates a portion that still is or remains to be captured or imaged such that the second portion is uninspected or uncaptured;
moving the imaging apparatus or system or the robotic or manual ureteroscope to search for any one or more residual fragments; and
in a case where any residual fragment(s) are found, removing the fragment(s).

24. A non-transitory storage medium storing at least one program to be executed by a processor to perform a method for performing lithotripsy, where the method comprises:
obtaining a Computed Tomography (CT) scan;
segmenting an object, target, or sample;
starting lithotripsy;
inserting an Electro Magnetic (EM) sensor into a tool channel, or disposing the EM sensor at a tip of a catheter or probe of a robotic or manual ureteroscope or an imaging apparatus or system;
performing registration;
starting visualization of a viewing area;
moving the imaging apparatus or system or the robotic or manual ureteroscope to search for a urinary stone in an object or specimen;
crushing the urinary stone into fragments using a laser inserted into the tool channel;
removing the fragments of the urinary stone using a basket catheter;
inserting the EM sensor into the tool channel, or disposing the EM sensor at the tip of the catheter or probe of the robotic or manual ureteroscope or the imaging apparatus or system;
performing registration;
starting visualization of a viewing area;
displaying a first portion and a second portion of the viewing area, where the first portion indicates a portion that has been captured or inspected and the second portion indicates a portion that still is or remains to be captured or imaged such that the second portion is uninspected or uncaptured;
moving the imaging apparatus or system or the robotic ureteroscope to search for any one or more residual fragments; and
in a case where any residual fragment(s) are found, removing the fragment(s).
